(19) United States
(12) Patent Application Publication
(10) Pub. No.: US 2006/0014178 A1
(43) Pub. Date: Jan. 19, 2006

(54) Use of Mrf-2 for screening and therapies

(76) Inventors: Robert H. Whitson, Pasadena, CA (US); Keiichi Itakura, Arcadia, CA (US)

Correspondence Address: PERKINS COIE LLP, POST OFFICE BOX 1208, SEATTLE, WA, 98111-1208, US

(21) Appl. No.: 11/140,510

(22) Filed: May 25, 2005

Related U.S. Application Data
(60) Provisional application No. 60/574,906, filed on May 26, 2004.

(51) Int. Cl. C12Q 1/68; C12P 19/34

(52) U.S. Cl. 435/6; 435/91.2

(57) ABSTRACT

Mrf-2 is essential for accumulation of lipid stores in postnatal life. Homozygous loss of the ARID gene Mrf-2 resulted in a high rate of neonatal mortality that was partially strain-dependent in mice. Loss of Mrf-2 expression did not affect embryonic survival, embryonic growth or birth weight. Lipid accumulation was severely reduced in brown adipose of Mrf-2^{-/-} neonates at 24 hours of age, however, and Mrf-2^{-/-} mice weighed significantly less than controls from postnatal day five onward. Adult Mrf-2^{-/-} mice were lean, with significant reductions in brown and white adipose tissues, and in the percentage of body fat. Mrf-2^{-/-} and Mrf-2^{±} mice were also resistant to weight gains and obesity when maintained on high fat diets.

### CROSS-REFERENCE TO RELATED APPLICATIONS

This application claims the benefit of U.S. Provisional Application No. 60/574,906, filed May 26, 2004, which is herein incorporated by reference in its entirety.

### BACKGROUND

Each of the references cited herein is incorporated by reference in its entirety. A complete listing of the citations is set forth at the end of the specification.

Mrf-2 is a member of the ARID (AT-rich interaction domain) family of transcription factors. The ARID is a DNA-binding motif found in proteins with diverse functions. ARID proteins exist in many types of living organisms, from species that include plants and fungi as well as animals [1-4]. The ARID has a unique structural motif that comprises 6-8 helices [4, 5]. In many proteins, the ARID recognizes short DNA sequences (5-6 bp), either alone, or in combination with other DNA-binding domains [1, 2, 6, 7]. Other ARID-containing proteins recognize cruciforms or other structures that contain single-stranded DNA [8, 9]. The structure-specific ARID proteins are subunits of “chromatin remodeling” complexes such as SWI/SNF or Brahma [3, 8, 9]. Many of the sequence-specific ARID proteins modulate gene expression at key steps in differentiation [1-3, 6].

Mrf-2 was first identified by its ability to bind to multiple DNA sequences in the major immediate-early promoter/enhancer/modulator of human cytomegalovirus (CMV) [10]. Binding site selection studies and other evidence indicate that the Mrf-1 and Mrf-2 ARID peptides bind with high affinity to the target sequence AATA[T/C] [7]. Experimental analysis of purified peptides encompassing the DNA-binding domain of Mrf-2 indicates that the DNA binding domain is probably conserved in all members of this protein family [4]. Mrf-2 and Mrf-1 (which has a nearly identical ARID to Mrf-2) have been cloned by screening an expression library for proteins that recognize specific viral DNA sequences [7, 10]. Although indirect evidence suggests that these proteins affect viral transcription, their normal cellular functions in animals have not been elucidated to this point. Understanding of the function of Mrf-1 and Mrf-2 is important, particularly if their functioning affected a widespread human health issue, such as obesity or diabetes.

Obesity, often described as a dysregulation of energy balance, is rapidly overtaking smoking as the leading preventable cause of death in the United States. The health problems associated with obesity have the potential to cripple the U.S. economy with lost productivity and increased medical care costs. At the same time, the demand for weight-loss treatments has fueled a multi-billion dollar industry. Therefore, novel, safe and effective anti-obesity treatments will find a ready market and are needed urgently.

The problems identified in the prior art are not all the problems in the prior art. The foregoing examples of the related art and limitations related therewith are intended to be illustrative and not exclusive. Other limitations of the related art will become apparent to those of skill in the art upon a reading of the specification and a study of the drawings.

### SUMMARY

The AT-rich interaction domain (ARID) protein Mrf-2 is essential for the accumulation of triglycerides in early postnatal and adult life in animals. Thus, a lack of Mrf-2 is implicated in a higher rate of neonatal mortality or, in an animal that survives to adulthood, a lack of adipose tissue and leanness throughout its life. Mrf-2 also affects craniofacial development, obesity, diabetes, and inborn errors in metabolism. The experimental data described herein shows that mouse strains lacking Mrf-2 expression result in high rates of neonatal mortality, slower neonatal weight gain, significant reductions in adult weight and adiposity, and potential craniofacial defects.

Thus, various screening methods for Mrf-2 to detect the genotype and/or phenotype associated with a presence or absence of Mrf-2 are contemplated. The screen can be administered at any time, from the prenatal period to adulthood, to determine the genotype of Mrf-2 (wild-type^{+/+}, heterozygous^{±}, or homozygous recessive^{-/-}), alterations of the genotype such as deletions, truncations or mutations, and/or phenotypes associated with the presence or absence of Mrf-2. Also taught are methods of screening for compounds that modulate Mrf-2 in connection with treating conditions associated with a presence or absence of Mrf-2. Methods of using modulators of Mrf-2 activity to treat insulin sensitivity, obesity, excessive leanness, diabetes, triglyceride imbalance, craniofacial defects, and inborn errors in metabolism are also disclosed. These modulators may include proteins and polynucleotides, such as siRNA. These and other aspects are further elucidated in the detailed description.

In addition to the exemplary aspects and embodiments described above, further aspects and embodiments will become apparent by reference to the drawings and by study of the following descriptions.

### BRIEF DESCRIPTION OF THE DRAWINGS

FIG. 1 generally shows disruption of the mouse Mrf-2 gene. FIG. 1A is a map of the wild-type Mrf-2 gene (top), a map of the double-selection targeting vector (middle), and a map of the recombinant gene (bottom). Exon V of the Mrf-2 gene is indicated by the open box with a Roman numeral V. The neomycin positive-selection cassette and the thymidine kinase negative-selection cassette are shown as shaded and open boxes, respectively. The probe used for Southern blotting is represented as a black box, and the expected EcoR1 or BamH1/EcoR1 fragments are shown as solid lines. The PCR primers used to detect the Neo^{r} marker are shown as solid arrowheads, and the primers used in the PCR assay shown in FIG. 1D are shown as open arrowheads; the PCR products are depicted as dotted lines.

FIG. 1B is two Southern blots of the four positive ES cell lines. DNA was isolated from ES cell lines, digested with BamH1 and EcoRI, and analyzed by Southern blotting with the probe shown in FIG. 1A (left blot) or an exon V probe (right blot).

FIG. 1C is a Southern blot analysis of an F2 mouse litter. DNA was treated and analyzed as in FIG. 1B. The positions of the wild-type EcoR1 fragment and the recombinant BamH1/EcoR1 fragment are indicated by the open and closed arrowheads, respectively, as in FIG. 1B. The genotypes of the parents and their offspring are indicated at the top of the blot.

FIG. 1D is a gel showing PCR analysis of F2 mice. DNA was isolated from toe samples, and analyzed by PCR using the primers indicated by open arrowheads in FIG. 1A. The wild-type and recombinant PCR products are indicated by arrowheads as in FIG. 1B. The genotypes of the mice are shown at the top of the gel.

FIG. 1E is a Northern blot and a corresponding bar graph showing Mrf-2 expression in heart tissue. Total RNA was isolated from heart muscle samples from six Mrf-2^{+/+} mice, six Mrf-2^{±} mice and three Mrf-2^{-/-} mice, and analyzed by Northern blotting. The left-hand panels show sequential hybridization of a blot containing samples from six of the mice, with genotypes indicated at the top. The blot was hybridized successively to probes for Mrf-2 (upper panel) and cyclophilin (lower panel). A second Northern blot was performed with RNA samples from the remaining nine mice (not shown). For each RNA sample, the expression of Mrf-2 relative to cyclophilin was normalized to the average wild-type values on the same blot. The bar graph depicts the combined values from both blots.

FIG. 2 is a series of graphs demonstrating that Mrf-2^{-/-} mice have slower neonatal weight-gains and reduced adult weights, but normal embryonic growth. FIGS. 2A-2D are postnatal growth curves for normal and Mrf-2 deficient mice. Growth curves are shown for females in FIGS. 2A and 2C, and for males in FIGS. 2B and 2D. Solid circles and dashed lines represent Mrf-2^{+/+} mice, open boxes and solid lines represent Mrf-2^{±} mice and open circles and dashed lines represent Mrf-2^{-/-} mice. Symbols and error bars represent means ±SE for 5-8 determinations at each time point. FIG. 2E is a bar graph of the lengths of males and females (mixed) at 16-19 days of age. FIGS. 2F and 2G represent the lengths of males and females, respectively, at 17 weeks of age. FIG. 2H is a line graph showing prenatal weight gains for wild-type and Mrf-2-deficient mice. The regression lines for all three groups superimpose. Symbols are the same as in parts FIGS. 2A-2D. FIG. 2I is a bar graph showing the weights of newborn pups, measured in the first 24 hours of life. The * symbol indicates significant differences from wild-type values, P<0.05.

FIG. 3 is a series of bar graphs demonstrating that Mrf-2^{-/-} mice have specific reductions in adipose tissue. FIG. 3A shows gonadal fat pad weights in adult males and females. Bars represent means, ±SE for 9-17 individuals of each genotype. The * and ** indicate significant differences between Mrf-2^{+/+} and Mrf-2^{-/-}, P<0.03 and 0.01, respectively. The † indicates significant differences between Mrf-2^{±} Mrf-2^{-/-31} , P<0.001. FIG. 3B shows body composition and organ weights were measured in an age-matched cohort of female mice. All tissue weights are normalized to total body weight. Bars represent means ±SE for six Mrf-2^{+/+} mice, six Mrf-2^{±} mice and four Mrf-2^{-/-} mice. The * indicates a significant difference from wild-type levels, P<0.05.

FIG. 4 shows morphological anomalies in adipose tissues from Mrf-2^{-/-} mice. In FIGS. 4A-4C, the photomicrographs of adult females depict fixed and stained sections. FIG. 4A is intrascapular brown adipose tissue (BAT) at 40× magnification. FIG. 4B is ovarian white adipose tissue (WAT) at 20× magnification. FIG. 4C is inguinal WAT at 20× magnification. Stained sections of fat tissue were prepared and analyzed to determine mean diameters of 200-300 lobules in each section. For each sample, the frequency distribution of lobule diameters was calculated, and the average frequencies for each group are shown in the line graphs. Symbols are the same as in FIG. 2, and represent means ±SE. The right-hand panels show the results of nuclei counts for the same sections. The * symbol indicates significant differences between wild-type and Mrf-2^{-/-} mice, with P values ranging from <0.001 to 0.05. FIG. 4D is a series of three photomicrographs showing intrascapular BAT (40× magnification) from pups that were euthanized at 24 hours of age. Bars at the bottom of the photomicrographs represent 50 μM in FIGS. 4A-4C and 25 μM in FIG. 4D.

FIG. 5 is a series of images illustrating that Mrf-2-expressing retroviral vectors stimulate adipogenesis in wild-type and Mrf-2^{-/-} mouse embryo fibroblast cultures. Wild-type and Mrf-2^{-/-} fibroblasts were treated with retroviral vectors that express Mrf-2A, Mrf-2B, Mrf-1 or Neomycin (the parent vector) as indicated. After three days, an adipogenic mixture was added, and the cultures were maintained in this medium for an additional 12 days. The cultures were then fixed and stained with Oil Red O to reveal the presence of lipid droplets, then stained with antibodies to Mrf-2 (the upper three panels in each group) or Mrf-1 (bottom panels). Photomicrographs (4× magnification) were then made using phase-contrast, red-fluorescence (for Oil Red O) or green fluorescence (antibody staining).

FIG. 6 is a series of six images illustrating that Mrf-2B reduces the requirement for insulin and dexamethasone in in vitro adipogenesis. Wild-type Mouse embryo fibroblasts were seeded at low density and treated with nothing, or with a retroviral vector that expresses Mrf-2B. The cells were incubated for three days, then treated with adipocyte induction media in which one of insulin, dexamethasone or IBMX were present at one-tenth the usual concentration, and the other two components were present at full strength. Adipocytes appear as dark spots in these brightfield photomicrographs, which were taken after 9 days of treatment.

FIG. 7 is a chart indicating that plasma leptin levels do not correlate with plasma triglyceride levels in Mrf-2^{-/-} mice. Leptin and triglyceride levels were measured in the same plasma samples from wild-type (filled circles) and Mrf-2^{-/-} mice (open circles).

FIG. 8 is a bar chart indicating reduced expression of Mrf-2 after addition of Mrf-2 siRNA. Mrf-2-c1 is a control showing expression without the addition of Mrf-2 siRNA and Mrf-2-3 shows about 80% decreased expression with the addition of Mrf-2 siRNA.

FIG. 9 is a bar chart indicating increased expression of Mrf-1 after addition of Mrf-2 siRNA. Mrf-1-c1 is a control showing expression without the addition of Mrf-2 siRNA and Mrf-1-5 shows about 100% increased expression with the addition of Mrf-2 siRNA.

FIG. 10 is a line graph showing cell growth in the absence of serum. siMRF-2 is the growth curve of the cells (Mrf-2-3) in which Mrf-2 expression is inhibited. The other growth curve is the negative control.

### DETAILED DESCRIPTION

A first aspect is a screen for the genotype of the Mrf-2 gene or for the presence or absence of the Mrf-2 protein for determining predisposition or genetic contribution to obesity, leanness, inborn errors in metabolism (IEM), craniofacial defects, and/or diabetes.

Obesity is an increase in body weight resulting from an excessive accumulation of fat in the body in comparison to lean muscle mass. Obesity has been defined as having a body mass index of 30 or greater. Leanness is a significant reduction in the percentage of body fat as compared to an expected amount of body fat for a particular subject. Experimentally, this can be determined as a reduction in the weights of specific fat depots, compared to the weights of other organs. In the mouse, the principal white adipose depots are inguinal, gonadal and retroperitoneal. The principal brown adipose depot is intrascapular. In Mrf-2^{-/-} mice, the weights of these fat pads, normalized to total body weight, are significantly lower than in wild-type mice. By contrast, the normalized weights of most other organs (such as the kidney, heart, or liver) are the same as in wild-type mice. Therefore, there is a specific reduction in fat. Another approach in determining leanness is to measure the percentage of total body fat, which may occur by simple chemical methods (i.e., fat extraction from dried carcasses) or by NMR or MRI. This aspect uses a radioactive tracer method that relies on the fact that there is a constant relationship between the total body water and lean body mass.

Inborn errors in metabolism are generally categorized into cellular intoxication, energy deficiency, and “mixed type.” Cellular intoxication disorders poison cells with excess precursors or alternate products. Energy deficient disorders deprive cells of necessary energy for proper functioning. Mixed type disorders combine pathology of both cellular intoxication and energy deficiency disorders. Generally, inborn errors in metabolism include the following defects: defective proteins, such as oxygen-carrying proteins, connective tissue protein, and clotting factors, defects in carbohydrate metabolism, defects on cholesterol and lipoprotein metabolism, mucopolysaccharide and glycolipid diseases, defects in amino and organic acid metabolism, porphyrias and bilirubinaernias, errors in fatty acid metabolism, defects in nucleotide metabolism, disorders in metal metabolism and transport, defects in peroxisomes, and defects associated with defective DNA repair.

“Craniofacial defects” is a term used to describe skull and facial deformities. Examples of craniofacial defects are abnormal skull shapes, malposition of the orbits, facial asymmetry, and dental defects.

Diabetes is a disease characterized by increased sugar levels in the blood, which can be a result of decreased insulin levels, resistance to insulin, or both.

Mrf-2^{-/-} genotype and some mutated Mrf-2^{+/+} and Mrf-2^{±} genotypes lead to leanness, craniofacial defects, inborn errors in metabolism, and a decreased risk of diabetes. Thus, a screen showing that a subject has a nonfunctional genotype, such as the Mrf-2^{-/-}, or a deletion, truncation, mutation or other genetic alteration of Mrf-2^{+/+} or Mrf-2^{±}, would indicate that the subject has a high probability of exhibiting these traits. Conversely, if the screen for the Mrf-2 genotype discovered that a subject had functional Mrf-2 genotype, such as Mrf-2^{+/+} or Mrf-2^{±}, then the subject would likely be of a normal to obese weight and have a lower risk of craniofacial defects and inborn errors in metabolism. However, this subject would be at an increased risk of diabetes as compared to a subject with a nonfunctional Mrf-2 genotype. As used herein, “subject” is any animal, including a mammal and a human. Candidates for such screening would include humans with craniofacial defects who are extremely lean, but have normal intelligence and/or lack mutations in Creb-bp.

The Mrf-2 screen may be directed toward the Mrf-2 gene itself, a Mrf-2 gene linked to a reporter gene, or the Mrf-2 protein. If the screen is directed to determining whether the subject has the functional or nonfunctional Mrf-2 genotype, such as by screening for a wildtype Mrf-2 gene, heterozygous Mrf-2 gene, or homozygous recessive Mrf-2 gene, the screen uses nucleic acid from the subject. The nucleic acid is DNA or MRNA. The nucleic acid is then examined for the Mrf-2 polymorphism.

Next, the nucleic acid is amplified using polymerase chain reaction (PCR) and primers described herein to detect the polymorphism. PCR is a system for the amplification of nucleic acid in vitro, which involves creating primers complementary to the target area of nucleic acid (here, the Mrf-2 gene). The primers, target nucleic acid, a heat-stable DNA polymerase (such as Taq) and excess nucleotides are subjected to thermal cycling. In each cycle, the target nucleic acid is denatured, annealed to the primers, and copied, thus creating exponential amplification of the Mrf-2 sequence.

The amplified DNA may then be sequenced to determine the genotype of Mrf-2. Alternatively, the detection process may comprise hybridizing the nucleic acid sample from a subject with equimolar amounts of labeled oligonucleotide probes unique to wild-type and mutant Mrf-2 sequences under conditions that permit specific hybridization of each to its target sequence. Then, the method quantifies the extent of hybridization of the two probes to molecules present in the nucleic acid sample. If there is no or very little hybridization of the wild-type probe, then there is homozygosity for the mutant allele (Mrf-2^{-/-}). If there is no or very little hybridization of the mutant probe, then there is homozygosity for the wild-type allele (Mrf-2^{+/+}). If there is roughly equal hybridization of wild-type and mutant probes, the subject is heterozygous for the two Mrf-2 alleles (Mrf-2^{±}). Another embodiment contemplates running the amplified nucleic acid product from a subject onto a gel to detect the varying bands known to be associated with various forms of Mrf-2, such as with single-strand conformation polymorphism analysis.

In yet another embodiment, the amplified nucleic acid may also be inserted into a vector and cloned, wherein the failure to detect more than one sequence from among the clones resulting from a given sample is indicative of homozygosity at that locus. The presence or absence of a Mrf-2 gene can be examined by cloning the Mrf-2 gene into a construct where the Mrf-2 gene is operably linked a reporter gene, such as chloramphenicol acetyltransferase, which will appear when the Mrf-2 gene is functional (Mrf-2^{+/+} or Mrf-2^{±}), but not when the gene is nonfunctional (Mrf-2^{-/-} or a genetic alteration of a normally functioning gene, such as with a truncation, missense mutation, splicing mutation, deletion, or other genetic error). A reporter gene is operably linked to a gene of interest when the reporter gene is coupled to the upstream sequence of the Mrf-2 gene. The reporter gene can then be used to see whether the Mrf-2 gene is transcribed. The reporter gene further shows which factors activate response elements in the upstream region of the Mrf-2 gene. The construct containing Mrf-2 and the operably linked reporter gene is then transfected into cultured cells. The cells are assayed for the presence of the reporter gene, the presence of which indicates that the subject has the functional form of the gene and absence of which indicates that the subject has the nonfunctional form of the gene.

It may be possible to distinguish between Mrf-2^{+/+} and Mrf-2^{±} form of the gene from the level of expression of the reporter. The phenotypes associated with Mrf-2^{+/+} were more pronounced than with Mrf-2^{±}. Thus, it is likely that the reporter gene operably linked to the Mrf-2^{+/+} gene will be expressed more strongly than the reporter gene linked the Mrf-2^{±} gene.

Another aspect screens a subject at the level of the Mrf-2 transcription factor protein and looks for the presence or absence of the protein in conjunction with screening for the various diseases and disorders described above. Screening for the presence or absence of Mrf-2 may use a cell-based screen. The specimen may be adipose tissue, other tissues, blood, lymph, or urine. The screening techniques may be any commonly known in the art for detecting the presence of proteins, such as Western blotting, other binding assays and activity assays. The antibodies used in the Western blot are labeled with fluorescence or with radioactive isotopes.

The screen may be conducted at any stage of a subject's life. Determining when the screen should be conducted could be dictated by the age associated with the condition. For example, it is logical to screen for extreme leanness and craniofacial defects at a prenatal stage when the defect would not be obvious and knowledge of the defect would be helpful information. Given the poor survival of neonatal Mrf-2^{-/-} mice, it is likely that newborn humans that lack Mrf-2 expression would also be at high risk for acute metabolic stress. Therefore, the identification of homozygous Mrf-2 mutations in infants and children should be used to identify the heterozygous carriers of these mutations in their families. In utero testing of at-risk fetuses could then be used to formulate strategies for ameliorating metabolic stress in the critical neonatal period. If the proclivity toward extreme leanness is detected, a treating physician may plan for immediate post-natal treatment to keep the subject warm and nourished and to reduce the chance of neonatal mortality.

If the screen were used to determine diabetes risk, on the other hand, the screen would likely be conducted in older subjects, such as subjects in adolescence or adulthood. Diabetes is a condition that is not usually immediately present or obvious upon birth, such as a craniofacial defect would be, and so would be applicable to a subject in any stage of life.

If the screen is conducted during the prenatal stage, the screened material is selected from amniotic fluid, blood, or tissue, but may be any sample from the subject comprising nucleic acid. If the screen is conducted postnatally, the screened material is selected from blood, urine, lymph, tissue, or any other material taken from the body that contains a sufficient amount of Mrf-2 nucleic acid or protein in order to conduct the screen. The material containing the nucleic acid may be obtained surgically, by swab, excretion, or any other reliable method.

An additional aspect is the use of Mrf-2 to treat insulin sensitivity, weight, diabetes, triglyceride imbalance, craniofacial defects, and/or inborn errors in metabolism in a subject. If Mrf-2 is lacking in a subject and causes, for example, extreme leanness, triglyceride imbalance, craniofacial defects, and/or inborn errors in metabolism, Mrf-2, a functional fragment of Mrf-2, and/or Mrf-2 agonists can be administered to a subject and/or stimulated within the subject to treat the condition. Mrf-2 agonists are any substance that will enhance, promote or stimulate the action of Mrf-2. Mrf-2 may be administered as in its protein or nucleic acid form. Mrf-2 production may also be stimulated within the subject such as with administration of vectors containing Mrf-2 nucleic acid.

If Mrf-2 is overexpressed or overabundant in a subject and causes, for example, obesity or diabetes, antibodies to Mrf-2, functional portions of Mrf-2 antibodies and/or Mrf-2 proteins or nucleic acid antagonists can be administered to a subject and/or stimulated within the subject to treat the condition. Mrf-2 antibodies include polyclonal and monoclonal antibodies, chimeric, single chain, and humanized antibodies, Fab fragments, or other immunoglobulin expression library. Mrf-2 antagonists are any substances that will nullify the action of Mrf-2. If the Mrf-2 antibodies, functional fragments thereof, or antagonists are administered to downregulate the effect of Mrf-2 protein or nucleic acid, the compound administered has specific binding affinity to its target, which is the Mrf-2 protein or nucleic acid target. Specific binding affinity means that compound binds to the target with greater affinity than it binds to other compounds.

The data from the Mrf-2^{-/-} mice is consistent with the theory that being lean is protective against type II diabetes. The Mrf-2^{-/-} mice have lower circulating levels of free fatty acids and triglycerides, which increases insulin sensitivity. Thus, the Mrf-2^{-/-} mice also have lower glucose levels, and higher insulin sensitivity in muscle. It is very clear, however, that the lean phenotype of Mrf-2^{-/-} mice arises from events that occur in the perinatal period.

Interfering with Mrf-2 activity in an adult animal would produce the same beneficial metabolic effects because adipogenesis continues in adult animals. The differentiation of fat cells in adults requires the same ordered expression of transcription factors as it does in embryos. Therefore, the discovery that in vitro adipogenesis is defective in Mrf-2^{-/-} mouse embryo fibroblasts (MEF's) reveals the unexpected finding that Mrf-2 deficiency has a similar effect in adults. Additionally, the loss of Mrf-2 activity in MEF's interferes with later developmental steps, particularly the accumulation of lipid stores. The fact that there is reduced expression of the late-stage adipogenic transcription factor C/EBPα in Mrf-2^{-/-} MEF's also shows that Mrf-2 is required for normal fat cell maturation. This is important because an adult stem cell may be further along the differentiation pathway than the embryo fibroblasts that are commonly used in these experimental studies.

The most potent drugs currently known for the treatment of type II diabetes are thiazolidinediones (TZD's), which act by binding to PPARγ. Because of its widespread effects in differentiation, PPARγ knockouts are embryonic lethal, but MEF's derived from PPARγ^{-/-} MEF's show the same defects in adipogenesis as Mrf-2^{-/-} MF's. PPARγ expression is normal in Mrf-2^{-/-} MEF's, which suggests that Mrf-2 acts downstream of PPARγ. Bone marrow precursors from adult PPARγ^{±} mice are also deficient in adipogenesis, but more proficient in osteogenesis, which indicates that the control of adipogenesis from adult stem cell precursors is similar to the control of adipogenesis from embryonic precursors. Thus, this aspect could be used to control diabetes and obesity in adult subjects.

For any method of treatment, the compound being administered is in a pharmaceutically acceptable carrier and in a therapeutically effective amount. A pharmaceutically acceptable carrier is non-toxic and compatible with other ingredients of the formulation. The carrier may contain additives such as substances that enhance isotonicity and chemical stability including buffers, antioxidants, polypeptides, proteins, polymers, sugars, and the like. Therapeutically effective amount is the amount of the compound that improves the condition being treated. A skilled clinician will determine the appropriate dosage, while taking into account the size, age, weight, and general physical condition of the subject. Administration may also be accomplished by any effective means including oral, parenteral, osmotic, or topical.

Another aspect screens for modulators of Mrf-2. The modulators may either agonize or antagonize Mrf-2 activity in a subject. The screen for a modulator of Mrf-2 comprises exposing Mrf-2 protein or nucleic acid to a test compound, determining whether the test compound binds to Mrf-2 protein or nucleic acid, and if it binds, selecting the test compound as a possible modulator of Mrf-2. The method may then further comprise determining the effect of the possible modulator on adipocyte maturation or adipocyte function in mouse embryo fibroblasts derived from Mrf-2^{-/-}, Mrf-2^{±} and Mrf-2^{+/+} embryos and, if the possible modulator affects adipocyte maturation or adipocyte function, determining the effect of the possible modulator on fat accumulation in adult Mrf-2^{-/-}, Mrf-2^{±} and Mrf-2^{+/+} mice. Mrf-2 DNA is used because DNA-binding is the most easily measured activity of Mrf-2, and this would provide the simplest method for high-throughput screening of candidate inhibitors.

The identification of candidate inhibitors may also be aided by the availability of the high-resolution structure for the Mrf-2 DNA-binding domain created by the present inventors. Numerous DNA-binding assays comprising the purified ARID peptide of Mrf-2 using well-established gel mobility-shift assays have been successfully performed. After testing a number of Mrf-1 variants, it was found that full binding activity is best achieved when the C-terminal portion is truncated to within 100 amino acids of the DNA-binding domain. This occurs because the C-terminal region of the protein exerts an inhibitory interaction with the DNA-binding domain, and relief of this inhibition requires interactions with other proteins, or post-translational modifications of the C-terminal region that do not occur when Mrf-1 is produced in bacteria or by cell-free translation. Similarly, the DNA-binding activity of full-length Mrf-2 is much lower than that of the isolated DNA-binding domain peptide, when both constructs are produced in bacteria. Although the inhibitory regions of Mrf-2 have not yet been identified, it is apparent that Mrf-2 also requires modifications or protein-protein interactions for full DNA binding activity.

Characterization of the factors that activate Mrf-2 binding could provide additional strategies for inhibiting the activities of Mrf-2. If, for example, Mrf-2 requires the presence of a co-factor protein to unmask its DNA-binding activity, compounds that interfere with this interaction would be effective in inhibiting Mrf-2 activity. Because there are a variety of methods for measuring protein-protein interactions in vitro, screening for such compounds would be relatively straightforward.

Modulating agents include Mrf-2-specific antibodies and small molecules. The modulating agent is specific to Mrf-2, meaning that it binds to or interacts directly with Mrf-2 polypeptide or nucleic acid and inhibits, enhances, or alters the function of Mrf-2. The modulating agent may contact a cell or cell lysate comprising Mrf-2. For screening the modulators in vitro, the Mrf-2 nucleic acid or polypeptide is at least partially purified. A kit for screening for a modulator of Mrf-2 in the search for modulating conditions of obesity, leanness, craniofacial defects and diabetes is also contemplated. The kit comprises Mrf-2 protein and instructions for use and also comprises tubes and reagents.

A related method for screening for a Mrf-2 inhibitor comprises the steps of incubating Mrf-2 protein in the presence or absence of a potential inhibitor. The test compound may be a Mrf-2 interacting protein that may bind directly to a Mrf-2 protein or may bind to a Mrf-2 substrate, binding partner, or cofactor. Then, the method determines the amount of Mrf-2 formed in the presence or absence of the test compound. Finally, the method compares the amount of Mrf-2 in the presence or absence of the test compound to a control sample without the potential inhibitor, wherein a decrease in the amount of product of Mrf-2 is indicative that the test compound is an inhibitor. Mrf-2^{-/-} embryo fibroblasts also provide a convenient in vitro system for screening potential Mrf-2 inhibitors. This screen could be accomplished by co-transfection assays as described herein. Native promoters for bona fide Mrf-2 target genes are used.

In another embodiment, short interfering RNA (“siRNA”) may be used to control expression of Mrf-2 by inactivating the Mrf-2 gene in a cell. siRNA disrupts the functioning of the Mrf-2 gene and acts to “knock out” the gene. siRNA is beneficial for shorter term control of a Mrf-2 because it eliminates the function of the gene but the loss of function is not heritable as a gene mutation or deletion would be. Thus, it allows specific control of the function of a gene.

Another aspect measures Mrf-2 activity based on its ability to “rescue” adipogenesis in Mrf-2^{-/-} embryo fibroblasts. This is accomplished by transfecting the cells with Mrf-2 expression plasmids (or infecting them with adenovirus that express Mrf-2). Then, the assay screens for Mrf-2 inhibitors by virtue of their ability to interfere with adipogenesis in the presence of Mrf-2.

The amount of Mrf-2 activity present in a sample after a test compound has been administered as compared to the Mrf-2 activity present in a control sample is determined by high throughput screening. Alternatively, the reduction in Mrf-2 can be quantified by spectrophometric analysis, optical density, or thin layer chromatography.

In order to identify the normal cellular functions of Mrf-2, mouse strains were propagated in which the Mrf-2 gene was disrupted and the Mrf-2 protein cannot be expressed [21]. Mrf-2^{±} mice appeared to be normal in most respects, but Mrf-2^{-/-} mice had a number of striking phenotypes. Mrf-2^{-/-} embryos develop normally, and have normal prenatal weight gains, but newborn Mrf-2^{-/-} mice are noticeably smaller than their wild-type or heterozygous littermates by 3-5 days of age. Mrf-2^{-/-} mice do gain weight as they mature, but remain 20-40% lighter than age- and sex-matched normal littermates throughout their lives. On the other hand, Mrf-2^{-/-} mice do reach 90% of normal lengths, and virtually all of the remaining length difference is due to the shortened skull.

Adult Mrf-2^{-/-} mice are significantly leaner than Mrf-2^{±} or Mrf-2^{+/+} mice, as evidenced by significant reductions in the weights of a number of fat depots, and significant reductions in the overall percentage of body fat (up to 70% lower). Microscopic examination of both brown and white adipose tissues in Mrf-2^{-/-} mice showed that there is a significant reduction in the amount of fat per cell, compared with wild-type controls. On a body-weight basis, Mrf-2^{-/-} mice consumed at least as many calories per day as wild-type mice. Unlike wild-type mice, however, Mrf-2^{-/-} mice did not gain weight or become obese when they were subjected to a high-fat diet, even though weight-normalized calorie intake remained the same in these two groups. Mrf-2^{-/-} mice have a very high neonatal mortality rate, and this is heavily strain-dependent: on a 129S1 genetic background, more than 98% of Mrf-2^{-/-} mice die before weaning; on a C57B1/6J genetic background, about 70% of Mrf-2^{-/-} mice die in this period.

Neonatal mortality in Mrf-2^{-/-} mice is due to their inability to accumulate fat. This is supported by microscopic examinations of brown adipose tissue in newborn Mrf-2^{-/-} mice, which showed a nearly-complete absence of lipid droplets. The accumulation of fat in brown adipose is essential for thermoregulation in neonatal mice, because they are an altricial species, meaning that they are unable to thermoregulate at birth. An essential step in the acquisition of thermogenic capacity is the expression of the mitochondrial uncoupling protein, UCP1. High levels of UCP1 in mature brown adipose tissue causes mitochondrial membranes to “leak,” and results in the uncoupling of fatty acid oxidation and the generation of ATP [23]. When this occurs, the electrical potential that normally accumulates in the form of a proton gradient across the mitochondrial membrane cannot be converted to chemical energy, and is lost as heat instead. In both mice and humans, brown adipose plays an essential role in thermogenesis in neonatal life, but while brown adipose disappears in adult humans, it persists in adult mice. As a result, thermogenesis was long thought to be of little relevance to human energy consumption. Recently, however, it was shown that human white adipocytes can take on the thermogenic characteristics of brown adipose when they over-express PGC-1α, a co-factor for PPAR-γ. [24]. This has important implications for the treatment of obesity and diabetes, because PPAR-γ is the target for thiazolidinones, which are currently the most potent treatment for type II diabetes. Alteration of energy balance through an increased utilization of fat in white adipose tissue can be used for the treatment of obesity.

It is known that primary mouse embryo fibroblasts (MEF's) can be induced to differentiate into adipocytes by the application of a hormone mixture, and it is believed that this in vitro differentiation process replicates the essential features of in vivo adipocyte maturation [25]. In vitro adipogenesis is significantly less efficient in fibroblast cultures that are derived from Mrf-2^{-/-} embryos; they produce significantly fewer mature fat cells, and the cells accumulate significantly less fat. Northern analyses of RNA isolated from Mrf-2^{-/-} and Mrf-2^{+/+} cultures were conducted at various time points in the differentiation process. Mrf-2^{-/-} cultures express normal levels of genes that are induced early in the differentiation process, but significantly lower levels of genes that are induced at later stages. These results indicate that Mrf-2 is essential for the later stages of adipogenesis, and particularly for developing the ability to accumulate fat. These results also demonstrate that Mrf-2-deficiency has “cell autonomous” effects in pre-adipocytes. This is important because it resolves that the altered gene expression in the tissues of intact Mrf-2^{-/-} mice is a direct effect of Mrf-2 deficiency and does not occur in response to changes in serum metabolites.

One of the genes whose expression is reduced in differentiating Mrf-2^{-/-} cultures is C/EBPα. C/EBPα is generally known to be essential for adipocyte maturation [26] and function in vitro and in vivo, and thus, reduced C/EBPα expression explains the defect in adipogenesis in Mrf-2^{-/-} MEF's. Examination of the promoter sequences in the mouse C/EBPα gene reveals the presence of a number of canonical Mrf-2 binding sites, and these are conserved in the human gene as well. These data indicate that C/EBPα may be a direct target for Mrf-2.

The data also indicate that Mrf-2-deficiency is protective against diabetes. Mrf-2^{-/-} mice have significantly lower levels of blood glucose in both the fasted and non-fasted state, and are more efficient than wild-type mice at clearing an oral glucose load. Also, both basal and insulin-stimulated glucose uptake is faster in skeletal muscle isolated from Mrf-2^{-/-} mice. Since insulin levels are not increased in Mrf-2^{-/-} mice, these mice are more insulin-sensitive. Mrf-2 deficiency likely has a direct effect on insulin sensitivity. Studies in the last decade have shown that, in addition to their importance in energy storage, adipocytes secrete a number of hormones that modulate the response to insulin in other tissues [25]. In general, fat-depleted adipocytes secrete hormones that increase insulin sensitivity, and fat-filled adipocytes secrete hormones, such as leptin, that blunt insulin sensitivity. Therefore, Mrf-2^{-/-} mice are more insulin sensitive because they are lean, and this is supported by the fact that serum leptin is significantly reduced in Mrf-2^{-/-} mice.

The rate of energy expenditure is abnormally high in Mrf-2^{-/-} mice and this finding is supported by a number of observations. Indirect calorimetry experiments show that overall energy consumption is significantly higher in Mrf-2^{-/-} mice than in wild-type mice. Mrf-2^{-/-} mice are not more active than wild-type mice are and the differences in energy consumption are greater during the light phase of the diurnal cycle, when mice are typically asleep. This indicates that Mrf-2^{-/-} mice have a higher basal metabolic rate than wild-type mice do. The enhanced rate of energy expenditure contributes to the failure of Mrf-2^{-/-} mice to accumulate body fat by depleting lipogenic substrates. This fact is supported by the observation that the concentrations of several serum metabolites (glucose, free fatty-acids and triglycerides) are significantly reduced in both fasted and non-fasted Mrf-2^{-/-} mice. In addition, Mrf-2^{-/-} mice have significantly elevated levels of serum lactate. This indicates that Mrf-2^{-/-} mice consume carbohydrates at an abnormally high rate, but fail to store this metabolic energy as fat.

Microscopic examination of the inguinal fat pads of Mrf-2^{-/-} mice has revealed the presence of large tracts of “multilocular” adipocytes, meaning that lipid is stored in multiple small vesicles, rather than a single large one. This morphology is more typical of brown adipose than white adipose. Ectopic expression of brown adipose in fat pads that usually contain only white adipose occurs when mice are subjected to cold stress [27]. The expression of UCP1, the hallmark of brown adipose, is not elevated in either brown fat or white fat of Mrf-2^{-/-} mice, but expression of UCP2 (a closely related uncoupling protein) is elevated in brown adipose. Further, PGC-1α expression and PPAR-γ expression are elevated in white adipose tissues of Mrf-2^{-/-} mice. These data are consistent with the finding that Mrf-2^{-/-} mice squander the metabolic energy generated by the oxidation of glucose through processes that are similar to those that occur in brown adipose.

Comparing the expression levels of a number of genes in the peripheral tissues of adult Mrf-2^{-/-} and Mrf-2^{+/+} mice, most of the observed changes are adaptive responses to the depletion of lipogenic substrates. In liver tissue of Mrf-2^{-/-} mice, for example, there is increased expression of enzymes that catalyze the rate-limiting steps in lipogenesis. These include fatty-acid synthase, stearoyl CoA desaturase (SCD1) and acyl CoA oxidase.

Ordinarily, these changes would be expected to increase the lipid content in liver, but liver triglycerides are significantly reduced in Mrf-2^{-/-} mice, even when they are maintained on a high-fat diet. The increase in SCD1 is particularly interesting, because this gene was recently identified as a key target for leptin in liver [28]. Leptin causes dramatic repression of SCD1 expression, which leads to depletion of liver triglycerides. The importance of this regulatory pathway is verified by the fact that SCD1 knockout mice are lean, and the absence of SCD1 reverses hepatic steatosis and increases the metabolic rate in leptin-deficient (ob/ob) mice, without affecting food intake [28]. Increased expression of SCD1 in Mrf-2^{-/-} mice is entirely consistent with the reduction in circulating leptin levels, but this is not sufficient to reverse either the depletion of liver triglycerides or the their hypermetabolism. Although it remains possible that Mrf-2 deficiency increases metabolism by affecting unidentified genes in peripheral tissues, the data indicate that the hypermetabolism of Mrf-2^{-/-} mice is a secondary effect of defects in lipogenesis.

Because the hypothalamus is a key regulatory center for both food consumption and energy expenditure, hypothalamic gene expression in Mrf-2^{-/-} and wild-type mice are also compared. Mrf-2^{-/-} mice express slightly higher levels of NPY, which is an orexigenic (hunger-inducing) neuropeptide, and lower levels of POMC, an anorexigenic (satiation-inducing) neuropeptide. Mrf-2^{-/-} mice also express higher levels of Vgf, a neuropeptide that decreases the metabolic rate. These changes are consistent with the fact that circulating leptin levels are decreased in Mrf-2^{-/-} mice, and would tend to increase, rather than decrease food consumption. They would also decrease, rather than increase energy expenditure. Therefore, they do not explain the lean phenotype of Mrf-2^{-/-} mice.

Mrf-2^{-/-} mice also have characteristic craniofacial anomalies that consist of a pronounced shortening of the skull along the longitudinal axis, compared to the transverse axis. The use of careful morphometric analyses of skull x-rays has found that other measurements of skeletal growth (such as tibial length) are normal in Mrf-2^{-/-} mice [36]. Although it has been suggested that the craniofacial anomalies contribute to the lean phenotype, there is no evidence to support this. The same combination of leanness and craniofacial anomalies is found in patients with Rubenstein-Taybi syndrome [29-30]. This results from mutation or heterozygous loss of the gene for Creb-bp, a protein that modulates the activities of the cyclic AMP response-element binding protein (CREB) [30]. The phenotypic similarities between Mrf-2^{-/-} mice and Creb-bp^{±} mice indicate that Mrf-2 and Creb-bp may act in some of the same pathways. Both mice and humans with Creb-bp mutations suffer from mental retardation, however [29], and it has been shown that both learning and memory are normal in Mrf-2^{-/-} mice. Thus, Mrf-2 and Creb-bp likely interact in some metabolic pathways, which is further supported by the known fact that CREB plays a role in adipogenesis [26, 30]. Because craniofacial malformations are the most common congenital defects in human beings, characterization of skeletal growth, histological examinations of bone, and the effects of Mrf-2 deficiency on in vitro osteogenesis are all important findings for elucidating mechanism of Mrf-2 deficiency in humans.

Another aspect determines whether Mrf-2 exerts direct control over C/EBPα expression. This determination exploits the Mrf-2^{-/-} MEF lines by creating a reporter plasmid that places the catecholamine acetyl transferase gene under the control of the mouse C/EBPα promoter. Then, the reporter plasmid is transfected into both Mrf-2^{-/-} and Mrf-2^{+/+} MEF cultures that have been treated with the adipogenic hormone mixture. An expression plasmid for Mrf-2 along with the reporter plasmid is co-transfected into the same cultures. The finding is that the activity of the reporter gene is significantly lower in the Mrf-2^{-/-} cultures, and increases significantly in the presence of the Mrf-2 expression plasmid.

Using the in vitro MEF differentiation assay, additional genes as candidate targets for Mrf-2 are also identified. RNA isolated from Mrf-2^{-/-} and Mrf-2^{+/+} cultures at various stages of differentiation is analyzed using microarrays, and genes with significant differences are analyzed in the same manner as C/EBPα.

It is also determined whether the lean phenotype of Mrf-2^{-/-} mice results primarily from the absence of Mrf-2 in adipose tissue. A series of mouse strains are propagated in which the Mrf-2 knockout occurs in single tissues. These may include white adipose, brown adipose, skeletal muscle and liver. Loss of Mrf-2 expression in adipose alone is sufficient to create a lean phenotype, and loss of expression in other tissues is not.

In order to assess the relative importance of adipogenic, metabolic and anorectic effects of Mrf-2 deficiency, the experiments described herein take advantage of a number of animal models in which the hypothalamic regulatory circuits are disrupted by chemical or genetic manipulations. Specifically, Mrf-2^{-/-} mice are crossed with ob/ob, db/db and agouti yellow (A^{y}/a) mice. Neonatal Mrf-2^{-/-} mice are treated with monosodium glutamate (MSG) and adult Mrf-2^{-/-} mice are treated with gold thioglucose (GTG).

MSG, GTG and the A^{y}/a, ob/ob and db/db mutations all lead to extreme obesity, but because they act by different mechanisms, comparing their effects in a Mrf-2^{-/-} genetic background is highly informative. A^{y}/a mice misexpress the agouti protein in the hypothalamus [31]. Normally, the agouti protein is expressed only in skin where it regulates coat color. Because the agouti protein resembles melanocortin, it blocks hypothalamic melanocortin receptors in the A^{y}/a mice. This suppresses the anorectic signaling pathways and leads to hyperphagia. GTG is toxic to glucose-sensitive neurons in the hypothalamus that control appetite, and like the A^{y} mutation, GTG treatment leads to hyperphagia [32]. MSG, which is a more potent neurotoxin than GTG, causes more extensive damage to the hypothalamus and also affects the sympathetic neurons that respond to signals from the hypothalamus. Unlike GTG treatment, MSG treatment does not lead to hyperphagia. GTG treatment does lead to obesity and this is believed to be due to the loss signaling from the hypothalamus to the sympathetic nervous system and a consequent decrease in the metabolic rate [32, 33]. Support for this hypothesis comes from the fact that GTG-lesioned mice have reduced GLUT4 expression and a decreased UCP1 response to cold stress in brown adipose, and lower body temperatures compared to untreated controls [33]. MSG-lesioned mice are also leptin-insensitive and have high circulating leptin levels [32, 33].

Leptin signaling is also impaired in ob/ob and db/db mice, which harbor mutations in the genes for leptin and the leptin receptor, respectively [34-38]. Because leptin exerts coordinate effects that decrease food intake and increase metabolism, deficits in both pathways contribute to obesity in ob/ob and db/db mice. Thus, when ob/ob mice are pair-fed with normal mice, their percentage of body fat remains significantly elevated, even though their weight decreases to normal levels [39]. Although mice with all of these deficits become obese, their responses to experimental manipulations are vastly different, and these differences serve to illuminate the mechanisms by which they act.

An illustrative example comes from studies that introduced these hypothalamic mutations into a Vgf^{-/-} genetic background. Vgf is a hypothalamic neuropeptide that regulates metabolism, but does not appear to exert direct effects on food intake [40]. A^{y}/a, Vgf^{-/-} mice are hyperphagic, but do not become obese because increases in their metabolic rate compensate for the increase in food intake. Vgf^{-/-}, ob/ob mice gain less weight than ob/ob mice, but still become obese. MSG-lesioned Vgf^{-/-} mice become almost as obese as MSG-lesioned wild-type mice. These studies indicate that Vgf regulates energy balance mainly by reducing energy expenditure, and acts at a point that is downstream of hypothalamic melanocortin receptors [35]. Similar experiments with Mrf-2^{-/-} mice allow for the identification of the regulatory circuits where Mrf-2 expression is essential, and understanding of the interrelationships between these circuits and other regulatory pathways.

Another aspect uses the Mrf-2^{-/-} MEF lines to determine whether Mrf-2 is essential for mCMV infection. Preliminary experiments involve performing in vitro infection assays on Mrf-2^{-/-}, Mrf-2^{±} and Mrf-2^{+/+} MEF lines to compare mCMV infectivity in these cell lines. Mrf-2-deficient lines showing an altered susceptibility to mCMV are tested for in vivo infectivity. Because CMV infections pose a deadly risk to bone-marrow transplant recipients, AIDS patients, and others with immune suppression, a finding that Mrf-2 plays a role in CMV infections is highly significant.

The absence of the Mrf-2 protein leads to a lean phenotype by interfering with fat cell maturation, by altering the basal metabolic rate, or by a combination of these effects. Further, the effect of Mrf-2 on inborn errors in metabolism, such as the effect that altering the basal metabolic rate has on these genetically-induced errors. In addition, the lack of Mrf-2 expression is protective against the development of diabetes, either by direct effects on insulin action, or as a side-effect of promoting leanness. Therefore, this aspect teaches a method to identify compounds that interfere with the normal actions of Mrf-2 as a means of developing anti-obesity and other drugs. Because adult mice that lack Mrf-2 remain relatively healthy, there is a strong probability of identifying Mrf-2 inhibitors with a minimum of deleterious side-effects for the treatment of humans.

Experimental Materials and Methods

Mice. Breeding stock (strains 129S1 and C57B1/6J) was obtained from Jackson Labs (Bar Harbor, Me.). All other mice were bred in the City of Hope Animal Resources Center under AALAC-approved conditions.

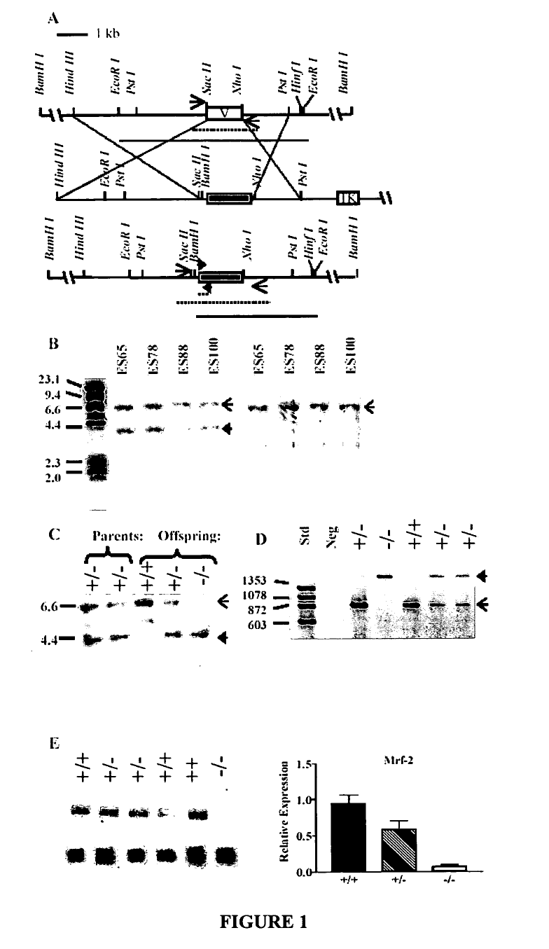

Targeted deletion of the Mrf-2 gene. In order to disrupt the Mrf-2 gene in ES cells, exon V was replaced with a neomycin-resistance cassette (Neo^{r}) via homologous recombination [11]. Exon V encodes the third and fourth helices of the ARID structure, which lie in its hydrophobic core. Since this core structure is conserved in all members of the ARID family, the loss of exon V is expected to disrupt the ARID structure [4]. A targeting vector (FIG. 1A) was constructed with the pKO Scrambler® system (Lexicon, The Woodlands, Tex.), and introduced via electroporation into embryonic stem (ES) cells derived from the 129S1 mouse strain. The 120 clones isolated using positive and negative selection [11] were evaluated by Southern blotting using an EcoRI/HinfI genomic fragment (FIG. 1A). The recombination event introduced a de novo BamH1 site, so that digestion of genomic DNA with EcoRI and BamHI produced fragments of 6.4 kb and 4.6 kb in wild-type cells and recombinants, respectively (FIGS. 1A-C). Four of the ES clones isolated by double selection (ES65, ES78, ES88 and ES100) had the intended disruption of the Mrf-2 gene (FIG. 1B), and these were used to produce 27 male chimeras.

The chimeras were mated to wild-type 129S1 and/or C57BL/6J females. Four chimeras from ES78 and three from ES88 were able to pass the Mrf-2 mutation to their offspring. Heterozygous males resulting from crosses to the C57BL/6J strain were also mated to wild-type C57B1/6J females to produce an N1/F1 generation. Brother×sister crosses were done with heterozygous F1, or N1/F1 mice, producing F2 or N1/F2 progeny with homozygous deletion of Mrf-2 exon V (FIGS. 1A, 1C and 1D). Northern blotting was used to confirm the loss of Mrf-2 expression by the absence of the 7.6 kb Mrf-2-specific transcript (FIG. 1E).

Survival rates for Mrf-2^{±} and Mrf-2^{-/-} mice were calculated on the assumption that the Mrf-2^{+/+} genotype was not deleterious for survival. By Mendelian principles, 50% of F1 mice and 25% of F2 mice should be Mrf-2^{+/+}, so the total number of mice without the deaths of Mrf-2^{±} or Mrf-2^{-/-} mice is given by T=Wt/0.5 (F1) or T=Wt/0.25 (F2), where Wt is the number of Mrf-2^{+/+} mice. Percent survival=100*(Ht/0.5T) for Mrf-2^{±} mice, and 100*(Kt/0.25T) for Mrf-2^{-/-} mice, where Ht and Kt are the numbers of surviving Mrf-2^{±} and Mrf-2^{-/-} mice, respectively.

PCR analyses. Pups were identified by a toe clipping code, and PCR analyses were performed on DNA samples isolated from toe digests [12]. Neo^{r} was detected using the following primers: 5′-CGCTTGGGTGGAGAGGCTATTCG-3′ (SEQ ID NO: 1) and 5′-CGGCAGGAGCAAGGTGAGATGAC-3′ (SEQ ID NO: 2). To determine the genotypes of mice in F2 and all subsequent generations, a PCR assay was developed with primers that flank exon V of Mrf-2. The forward primer is: 5′-TGCATAGAATGAATGACCCTGGTC-3′ (SEQ ID NO: 3); the reverse primer is: 5′-CGGAAGTGGACAGATGG-AATGG-3′ (SEQ ID NO: 4). The wild-type gene gives an 878 bp product, and the recombinant gene gives an 1816 bp product (FIG. 1D). Hotstar® Taq polymerase (Qiagen, Chatsworth, Calif.) was used in all PCR analyses.

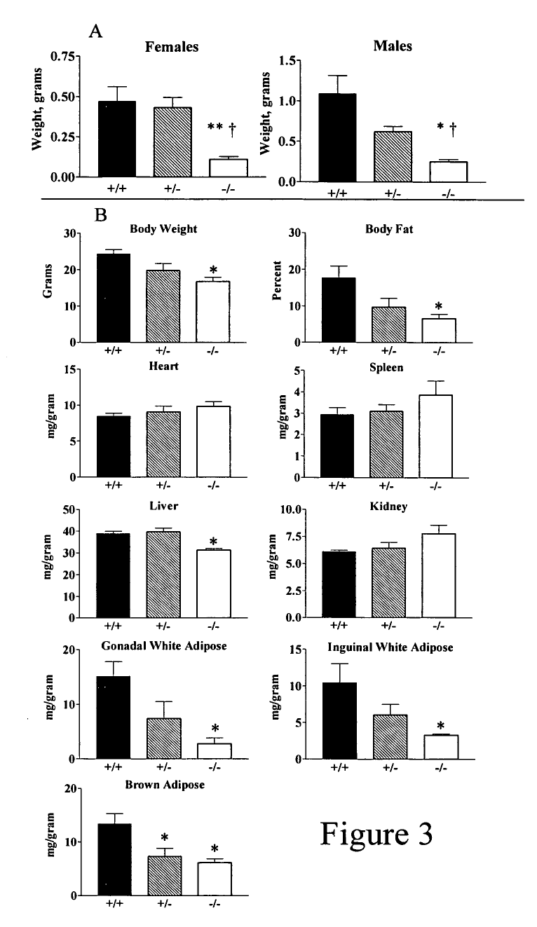

Analysis of adipose mass and body composition. These analyses were done on F2 (or N1F2) and subsequent generations of mice with mixed 129S1·C57BL/6J genetic backgrounds. For cross-sectional weight studies, animals from 5 to 200 days of age were weighed either in the morning or in the afternoon. To obtain the data shown in FIG. 3 and Table 1, animals were weighed, then anesthetized with an intraperitoneal injection of a mixture of ketamine and xylazine, (90 mg/kg and 10 mg/kg, respectively). Naso-anal lengths were then measured, and the anesthetized animals were exsanguinated by cardiac puncture. Epididymal or ovarian fat pads were then removed and weighed. Weights of contralateral fat pads from each mouse varied by less than 15%. WAT (white-adipose tissue) index (Table 1) is defined as the combined weights of the gonadal fat pads (mg), divided by total body weight (gm).

Percentage of body fat (FIG. 3, Table 1) was determined by calculating the dilution of injected ³H₂O, according to the method of Wolfe [13]. This method may underestimate total body water, but the maximum error is estimated at 5% [14]. For both male and female mice, the body-fat measurements correlated well with the WAT index (r2=0.758 and 0.707, respectively).

TABLE 1
Effects of standard and high-fat diets on adiposity in wild-type and Mrf-2-deficient mice.

| | | | Daily Calorie Consumption: | | | | | |
|---|---|---|---|---|---|---|---|---|
| Sex: | Diet: | Genotype: | Gross: | Normalized: | Weight Gain: | | WAT: | Percent Fat: |
| Males: | STD: | +/+ | 12.80 ± 1.23 | 0.39 ± 0.02 | −0.82 ± 0.67 | (6) | N.D. | N.D. |
| | | +/− | 10.59 ± 1.89 | 0.45 ± 0.02* | 0.39 ± 0.46 | (7) | N.D. | N.D. |
| | | −/− | 10.01 ± 0.60* | 0.48 ± 0.05* | 0.27 ± 0.26 | (8) | N.D. | N.D. |
| | High-Fat: | +/+ | 15.16 ± 0.48 | 0.35 ± 0.01 | 8.46 ± 2.45 | (5) | 51.0 ± 4.1 | 42.2 ± 1.6 |
| | | +/− | 14.50 ± 0.58* | 0.43 ± 0.01* | 0.70 ± 0.80* | (6) | 28.3 ± 6.4* | 18.5 ± 6.5* |
| | | −/− | 10.51 ± 0.45*† | 0.48 ± 0.02*† | −0.98 ± 0.57* | (6) | 13.5 ± 3.1*† | 23.7 ± 3.4* |
| Females: | STD: | +/+ | 14.64 ± 2.0 | 0.52 ± 0.07 | 0.61 ± 0.58 | (8) | N.D. | N.D. |
| | | +/− | 11.84 ± 0.40* | 0.45 ± 0.02* | 1.71 ± 0.92 | (11) | N.D. | N.D. |
| | | −/− | 10.92 ± 0.41*† | 0.53 ± 0.02† | 0.31 ± 0.43 | (8) | N.D. | N.D. |
| | High-Fat: | +/+ | 16.18 ± 1.11 | 0.43 ± 0.02 | 10.17 ± 3.14 | (6) | 36.3 ± 7.2 | 31.9 ± 2.8 |
| | | +/− | 12.48 ± 0.31* | 0.46 ± 0.01 | 2.17 ± 1.06* | (10) | 18.6 ± 1.8* | 16.7 ± 3.7* |
| | | −/− | 10.66 ± 0.38*† | 0.51 ± 0.01*† | −0.21 ± 0.86* | (8) | 8.2 ± 3.0*† | 10.6 ± 3.1* |

In Table 1, data for standard and high-fat diets are derived from the same cohorts of mice. Values for gross calorie consumption are given as kcal/mouse/day; values for normalized calorie consumption are given as-(kcal/mouse/day)/(gram body weight). Both values represent averages from 3-5 cages with 2 mice each, measured every 1-3 days for two weeks on the standard diet (10 determinations), and once or twice per week for 15 weeks on the high-fat diet (16 determinations). Data are expressed as means, ±SE. The value in parentheses in the weight-gain column indicates the number of mice used. P<0.01:*Mrf-2^{-/-} or Mrf-2^{±} versus sex- and diet-matched Mrf-2^{+/+}, † Mrf-2^{-/-} vs. sex- and diet-matched Mrf-2^{±}.

Morphological analyses of adipose tissues. Morphometric analyses of cell densities and fat lobule diameters were performed according to the methods of Cinti, et al. [15]. Intrascapular brown adipose tissue (BAT), and inguinal and ovarian WAT were fixed in freshly-prepared, saline-buffered paraformaldehyde. Paraffin sections were prepared and stained with hematoxylin and eosin by the Anatomic Pathology Laboratory at City of Hope. Lipid vacuole diameters and cell densities were analyzed in photomicrographs of representative sections using ImagePro Plus 4.0 software.

Feeding studies. Male and female mice, from 10-15 weeks of age were housed singly or in pairs in standard wire-topped cages. All animals were from the F2, N1/F2 or subsequent generations with mixed 129S1·C57B1/6J genetic backgrounds. The same mice were used sequentially for standard and high-fat feeding studies. Standard lab chow was Rodent Diet 5001 from LabDiet (PMI Nutrition International, Brentwood, Mo.). The nutritional density was 3.34 kcal/g, with 28.05% derived from protein, 12.14% from fat and 59.81% from carbohydrate. High-fat chow was Mouse Diet 9F (PMI). The nutritional density was 3.75 kcal/g, with 21.87% derived from protein, 21.6% from fat and 53.53% from carbohydrate. On both diets, the mice were fed ad libitum, and food pellets were weighed in the morning. The mice were weighed at the beginning and end of the standard chow feeding period, and weekly during the high-fat feeding period.

Analyses of embryos. All embryos were from the F2 or subsequent generations on the pure 129S1 genetic background. Gestational ages were timed from the appearance of a mating plug at 0.5 days post-coitus (dpc). Pregnant females were euthanized by CO₂ asphyxiation between 13.5 and 18.5 dpc. Embryos were removed from the yolk sacs, weighed, and fixed in 4% (w/v) saline-buffered paraformaldehyde overnight at 4° C. DNA was extracted from yolk sacs, then analyzed for genotype by PCR. To verify gestational ages, the embryos were evaluated by the Theiler criteria [16].

Statistical analyses. Data for the Mrf-2 genotypes were compared as groups using unpaired, two-tailed Student's t-tests. Groups that had unequal variances were compared using the Mann-Whitney t-test.

Experimental Results

Mrf-2^{-/-} mice have decreased viability that is partially strain-dependent. In the F1 generation, only 68.2% of the expected number of Mrf-2^{±} mice survived, suggesting that heterozygous disruption of the gene was moderately deleterious. The survival rate of Mrf-2^{±} mice improved slightly in the F2 and subsequent generations, as the trait was bred onto the C57B1/6J genetic background (66.1 % on the pure 129S1 background, 68.8% with a single cross to C57B1/6J, and 81.5% with an additional cross to C57B1/6J). Homozygous disruption of the Mrf-2 gene was even more deleterious, and this effect was also strain-dependent. On the pure 129S1 genetic background, only 6 of the 294 pups that survived long enough for genotype analysis were Mrf-2^{-/-}, which corresponds to a survival rate of only 6.3%. On the mixed 129S1·C57B1/6J genetic background, 42 of 713 pups were Mrf-2^{-/-}, which corresponds to an 18.8% survival rate. With an additional backcross to the C57B1/6J strain, 107 of 946 pups were Mrf-2^{-/-}, which corresponds to a 38.3% survival rate. There was no sex bias evident in any of the genotypes on either genetic background.

Because tissue for genotype analysis was taken from pups at 5-7 days of age, it was not immediately clear whether Mrf-2^{-/-} mice failed to develop in utero or died soon after birth. In order to address this question, we examined the survival of Mrf-2^{-/-} embryos on the 129S1 genetic background. The genotypes of 44 intact embryos in nine litters taken between 13.5 and 18.5 dpc were determined. The results indicated that the pre-natal survival rate for Mrf-2^{-/-} mice was as high as, or higher than that of wild-type or heterozygous littermates: 27.2% of the embryos were Mrf-2^{-/-}, 54.5% were Mrf-2^{±}, and 18.2% were Mrf-2^{+/+}. Examination of five Mrf-2^{-/-} pups that died within 24 hours of birth showed that they were grossly normal, and all of them had milk in their stomachs. Taken together, these data indicate that the Mrf-2^{-/-} mice underwent normal prenatal development, and survived long enough to begin suckling, but died within 24 hours of birth. Mrf-2^{-/-} mice that survived to adulthood appeared to be quite healthy, however, and several of them survived for well over a year.

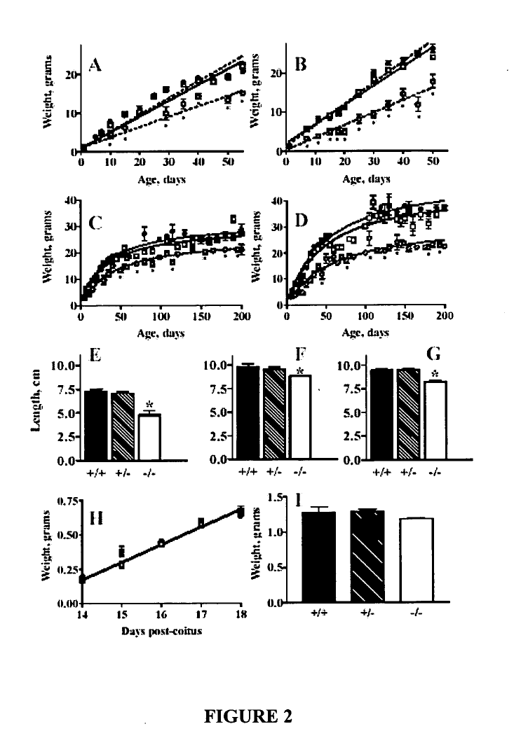

Mrf-2^{-/-} mice are lean. The most obvious phenotype associated with the surviving Mrf-2^{-/-} mice was a dramatic reduction in neonatal and adult weight gains (FIGS. 2A-D). Mrf-2^{-/-} pups were indistinguishable from their littermates at birth, but were noticeably smaller by five days of age. Mrf-2^{-/-} neonates did gain weight, but never achieved the weights of age- and sex-matched wild-type or heterozygous controls (FIGS. 2A-D). By contrast, Mrf-2^{±} mice had normal weight gains when maintained on normal lab chow (FIGS. 2A-D). Mrf-2^{-/-} mice were also substantially shorter than wild-type and heterozygous controls at early ages (FIG. 2E), but achieved 90% of wild-type lengths by 17 weeks of age (FIGS. 2F, 2G). These data indicate that in adult mice, Mrf-2 deficiency has a greater impact on weight gain than on overall growth. Surprisingly, loss of Mrf-2 expression did not inhibit weight gains in prenatal life. The weight-gains of Mrf-2^{-/-} embryos were comparable to those of Mrf-2^{±} and Mrf-2^{+/+} embryos from 13.5-18.5 dpc, and the weights of Mrf-2^{-/-} neonates were not significantly lower than those of controls when measured in the first 24 hours after birth (FIGS. 2H, 2I).

These data, coupled with the dramatic differences in weights at five days of age, indicate that newborn Mrf-2^{-/-} mice experience substantial metabolic stress. A variety of data indicated that adult Mrf-2^{-/-} mice were significantly leaner than age- and sex-matched Mrf-2^{±} and Mrf-2^{+/+} controls. One indication of this was the decrease in the weights of WAT fat pads in both males and females (FIG. 3A). In order to investigate this further, multiple indices of body composition were examined in age-matched cohorts of males and females. FIG. 3B shows that the percentage of body fat was significantly lower in the Mrf-2^{-/-} females. The figure also shows that the normalized weights of both WAT and BAT depots were significantly reduced, but the weights of non-adipose organs were not. The same trends were observed for Mrf-2^{-/-} males.

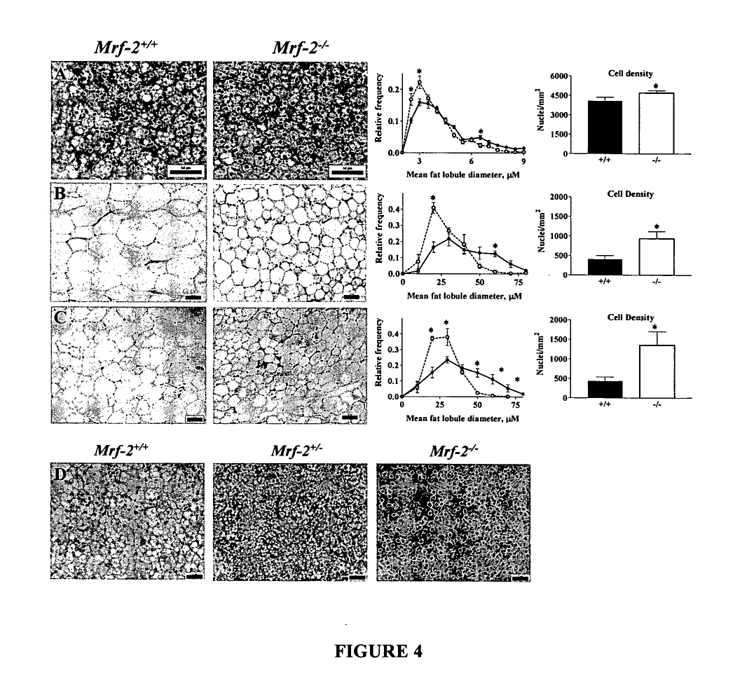

The reduction in fat depot weights appears to be due to reduced fat lobule size. FIGS. 4A-4C show that the mean fat lobule diameter was significantly reduced, while cell density was significantly increased in the intrascapular BAT depot and in the inguinal and ovarian WAT depots from Mrf-2^{-/-} females. These results imply that Mrf-2^{-/-} mice have less stored triglyceride per cell than wild type mice, as opposed to a decrease in the number of adipocytes. An inability to accumulate fat may contribute to the high rates of neonatal mortality in Mrf-2^{-/-} mice. This is suggested by microscopic examination of intrascapular BAT from mice that were euthanized 24 hours after birth. Lipid vacuoles were absent in BAT from Mrf-2^{-/-} neonates, but plentiful in BAT from their Mrf-2^{+/+} and Mrf-2^{±} littermates (FIG. 4D).

Reduced food intake does not appear to account for the lean phenotype of adult Mrf-2^{-/-} mice. In gross terms, Mrf-2-deficient mice did consume significantly fewer calories per day than age- and sex-matched Mrf-2^{+/+} mice (Table 1). When calorie consumption was normalized to body weight, however, the values for Mrf-2^{-/-} mice were actually somewhat higher than those of Mrf-2^{+/+} mice. To examine further the effects of diet on the lean phenotype of Mrf-2^{-/-} mice, adult mice of all three genotypes were subjected to a diet of high-fat breeder chow (21.6% of calories from fat, versus 12.1% for standard chow). After fifteen weeks on the high-fat diet, wild-type mice experienced significant weight gains. Mrf-2^{±} females also gained weight, but significantly less than the wild-type females. By contrast, Mrf-2^{±} males and Mrf-2^{-/-} mice of both sexes experienced no weight gains (Table 1). Wild-type mice became obese on the high-fat diet, as determined by two independent measurements (WAT index and percent body fat, Table 1). By contrast, neither the Mrf-2^{-/-} mice nor the Mrf-2^{±} mice became obese. Taken together, these data indicate that the lean phenotype of Mrf-2^{-/-} mice is not primarily due to differences in food intake. They also indicate that both heterozygous and homozygous disruptions of Mrf-2 are protective against diet-induced obesity.

Experimental Conclusions

The most striking phenotype of adult Mrf-2^{-/-} mice was leanness. Mrf-2^{-/-} adults weighed significantly less than age- matched and sex-matched controls (FIG. 2) and had tissue-specific decreases in both WAT and BAT (FIG. 3). Direct measurements of body composition confirmed that the percentage of body fat was significantly reduced in Mrf-2^{-/-} mice, whether they were maintained on standard lab chow or high-fat diets (FIG. 3, Table 1). The reduction in adipose tissue mass was due to a reduction in the lipid per cell amount, rather than a failure to produce adipose tissue, per se. Morphological examinations of intrascapular BAT, and inguinal and gonadal WAT revealed that the diameters of fat lobules in these tissues were significantly lower in Mrf-2^{-/-} mice (FIG. 4). Taken together, these data support the conclusion that Mrf-2^{-/-} mice have an impaired ability to accumulate triglycerides in adipose tissues. Although alterations in feeding behavior may contribute to the lean phenotype, they do not appear to be its primary cause. In absolute terms, adult Mrf-2^{-/-} mice consumed fewer calories per day than wild-type and heterozygous controls, but on a weight-normalized basis their calorie consumption was as high or higher (Table 1). This was true whether the mice were maintained on standard lab chow (12% fat), solid breeder chow (28% fat) or a high-fat liquid diet (35% fat—data not shown). When maintained on the high-fat breeder chow, Mrf-2^{+/+} mice experienced significant weight gains and became obese while Mrf-2^{-/-} mice did neither. Mrf-2^{±} mice that were maintained on breeder chow also had significantly lower weight gains, and significantly less adiposity than fat-fed Mrf-2^{+/+} mice. The data indicate that adult Mrf-2^{-/-} mice are lean, rather than dwarfed or runted.

Although Mrf-2^{-/-} mice are much shorter than wild-type mice at 16-19 days of age, this difference becomes smaller as the mice mature (FIG. 2). In contrast, the differences in weight are maintained or increased throughout adult life. Therefore, the more dramatic length differences in younger Mrf-2^{-/-} mice are primarily due to metabolic stress, rather than a direct result of Mrf-2 deficiency. Mrf-2^{-/-} mice had a very high rate of neonatal mortality. This appears to be heavily strain-dependent, ranging from nearly 100% on the pure 129S1 background, to about 60% with two backcrosses to C57B1/6J. The lean phenotype does not seem to be strain dependent, however, since the few Mrf-2^{-/-} mice that survived on the 129S1 background were also extremely lean. Pre-natal development appeared to be grossly normal in Mrf-2^{-/-} embryos, since their survival rates equaled or exceeded those of control embryos. The weight gains of Mrf-2^{-/-} embryos were also the same as those of wild-type littermates from 13.5 to 18.5 dpc, and the weights of newborn Mrf-2^{-/-} mice were nearly the same as those of wild-type littermates (FIG. 2). The effects of Mrf-2 deficiency become evident very soon after birth, however, so that Mrf-2^{-/-} mice weighed significantly less than wild-type and heterozygous littermates by five days of age.

Microscopic examination of intrascapular fat pads showed a complete absence of lipid vacuoles in BAT from Mrf-2^{-/-} neonates (FIG. 4). Since BAT is crucial for thermoregulation in neonates, it is possible that cold stress contributes to the high rate of neonatal mortality in Mrf-2^{-/-} mice. The data indicate that Mrf-2^{-/-} neonates and adults both fail to store fat in adipose tissue. The primary metabolic defect may arise in adipose tissue itself or increased metabolic rates in non-adipose tissues may deplete serum substrate pools for triglyceride synthesis. Phenotypic similarities between Mrf-2^{-/-} mice and other transcription factor knockouts show that Mrf-2 plays a role in adipocyte differentiation. Members of the CCAAT/enhancer binding protein (C/EBP) family are essential for adipogenesis, and C/EBPα knockouts, and C/EBPβ/C/EBPγ double-knockouts have high rates of neonatal mortality and severe reductions in fat pad weights in surviving adults [17-19]. The data also shows that hormone-stimulated adipogenesis is significantly inhibited in Mrf-2^{-/-} embryonic fibroblasts.

Mice that lack a single copy of the gene for CREB-binding protein (CBP) are also lean and resistant to diet-induced obesity [20]. Fibroblasts derived from CBP^{±} embryos are also deficient in hormone-stimulated adipogenesis, but triglyceride stores are apparently normal in BAT and WAT of CBP^{±} neonates [20]. This suggests that the link between defects related to in vitro adipogenesis and metabolic stress in neonates is not absolute. Use of the methods and use of transgenic mice clarify that relationship and show that Mrf-2 plays a role in the differentiation of multiple cells and tissues.

Over-expression of Mrf-2 stimulates adipogenesis. With the discovery that fibroblast cultures derived from Mrf-2^{-/-} mouse embryos have significant defects in adipogenesis, the next goal was to verify the role of Mrf-2 in adipocyte maturation by demonstrating that restoration of Mrf-2 expression rescues this phenotype. Retroviral vectors were constructed that express both the long and short splice variants of Mrf-2 (Mrf-2A and Mrf-2B, respectively). Then, both Mrf-2^{-/-} and Mrf-2^{+/+} mouse embryo fibroblasts were treated with these vectors and the cells were incubated for three days. Following this retroviral transduction, the cells were treated with a standard adipogenic hormone mixture consisting of insulin, dexamethasone and IBMX (a cyclic AMP phosphodiesterase inhibitor). After 12 days of this treatment, the cells were fixed, then stained with Oil Red O to reveal lipid droplets, then stained with antibodies to the C-terminal peptide of Mrf-2.

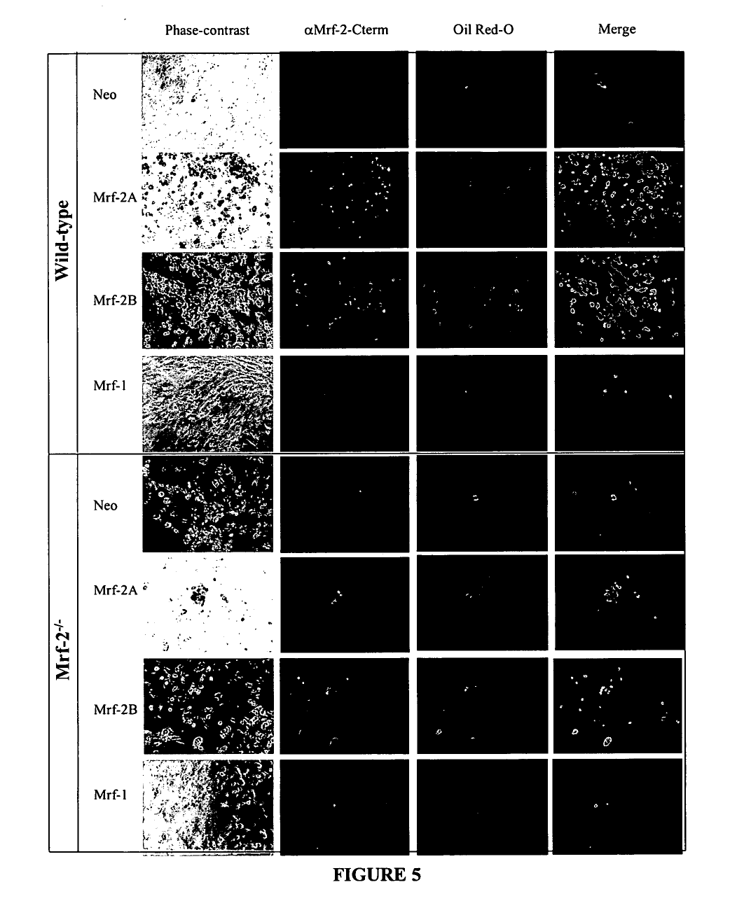

In four separate experiments, it was found that both Mrf-2A and Mrf-2B dramatically stimulated adipogenesis in both Mrf-2^{-/-} and Mrf-2^{+/+} mouse embryo fibroblast cultures (FIG. 1). The specificity of this effect was demonstrated by the absence of stimulation by either the parent retroviral vector, or by a retroviral vector expressing the closely-related ARID protein Mrf-1. Overexpression of Mrf-2 was not sufficient to stimulate adipogenesis in the absence of the adipogenic hormones, however. Antibody staining for Mrf-2 showed that the pattern of Mrf-2A or Mrf-2B expression correlated exactly with the pattern of Oil Red O staining (FIG. 5). Taken together, these results demonstrate that Mrf-2 is necessary, but not sufficient, for adipocyte development.

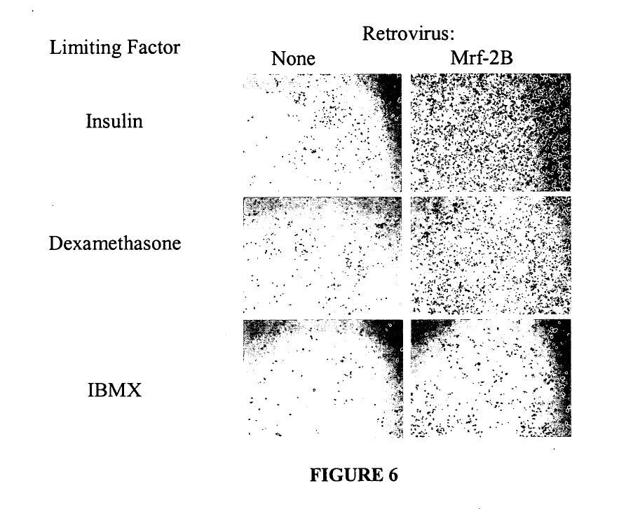

It has also been discovered that over-expression of Mrf-2 enhances the effects of insulin, dexamethasone and cyclic AMP. Mrf-2 is a downstream target for only the insulin and dexamethasone pathways, but not the cyclic AMP pathway. Mrf-2^{+/+} mouse embryo fibroblasts were transduced with the Mrf-2B retroviral vector. Then, the cells were treated with adipogenic mixtures in which the concentration of insulin, dexamethasone or IBMX was reduced, and the other two agents were maintained at their usual concentrations. It was found that Mrf-2B stimulated adipogenesis dramatically when insulin or dexamethasone was present at only one-tenth of their normal concentrations (FIG. 6). By contrast, Mrf-2B gave little or no stimulation when IBMX was limiting. Taken together, these results indicate that Mrf-2 co-stimulates the expression of adipogenic genes that lie downstream of insulin or dexamethasone signaling, but not genes that lie downstream of cyclic AMP signaling.

The positive relationship between insulin and Mrf-2 may seem surprising, in light of the fact that Mrf-2^{-/-} mice have improved glucose tolerance and increased insulin sensitivity. However this can be explained because selective knockout of the insulin receptor in adipose tissue is protective against obesity and obesity-related glucose intolerance (41). Adipocytes isolated from these Fat Insulin Receptor Knockout (FIRKO) mice showed a bimodal size distribution, with a significant increase in the percentage of small adipocytes. The smaller adipocytes expressed normal levels of PPAR-γ and GLUT4, which are considered to be markers of adipocyte differentiation, but had reduced expression of C/EBPα and FAS. These results suggested that insulin-signaling is required for adipocyte maturation, and that the arrest of this maturation process is protective against both obesity and diabetes. The phenotypes of Mrf-2^{-/-} mice are remarkably similar to those of FIRKO mice (21, 41). Like the adipocytes from FIRKO mice, our Mrf-2^{-/-} mouse embryo fibroblast cultures express normal levels of PPAR-γ, but reduced levels of both C/EBPα and FAS after stimulation with adipogenic hormones. Taken together, these data indicate that Mrf-2 works cooperatively with insulin-stimulated transcription factors to stimulate adipogenic gene expression.

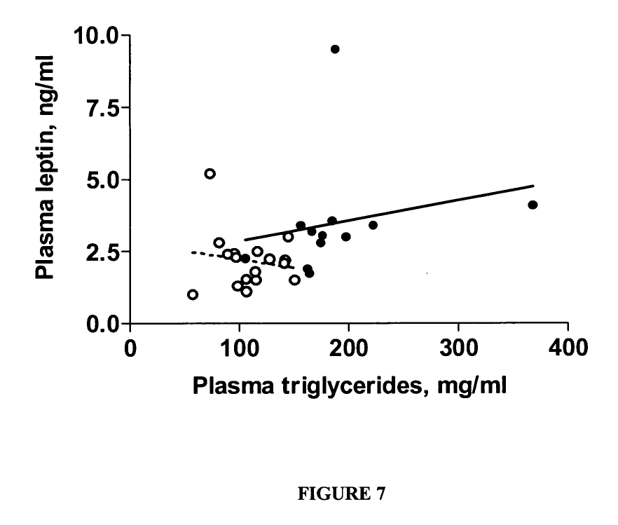

The presence of immature adipocytes has profound effects on energy balance because the immature fat cells send hormonal signals to the brain and to other peripheral tissues. It is becoming increasingly apparent that adipose tissue plays a significant role in the regulation of energy balance, via the regulated release of leptin, adiponectin, TNFα and resistin. Immature adipocytes favoring the release of insulin-sensitizing hormones (leptin and adiponectin or ACRP30) over diabetogenic hormones (resistin and TNFα) accounts for the effects of both the FIRKO and Mrf-2^{-/-} genotypes on peripheral tissues. The observation that the normal relationship between adiposity and leptin release is disrupted in both of these strains of knockout mice supports this finding (FIG. 7). Mrf-2^{-/-} mice are an important research tool for the study of adipose tissue and its effects on energy balance and obesity.

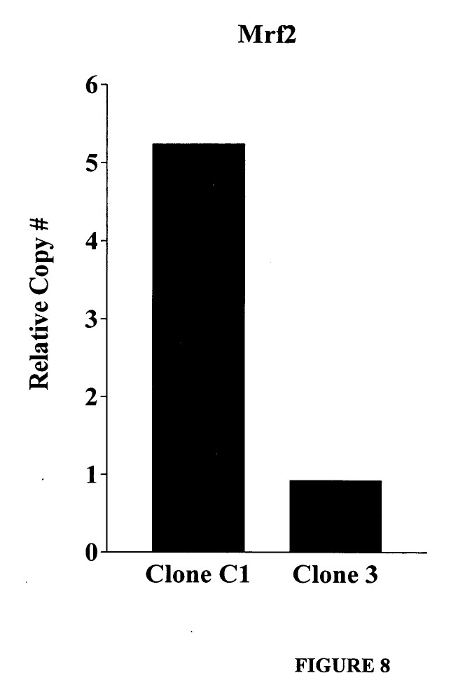

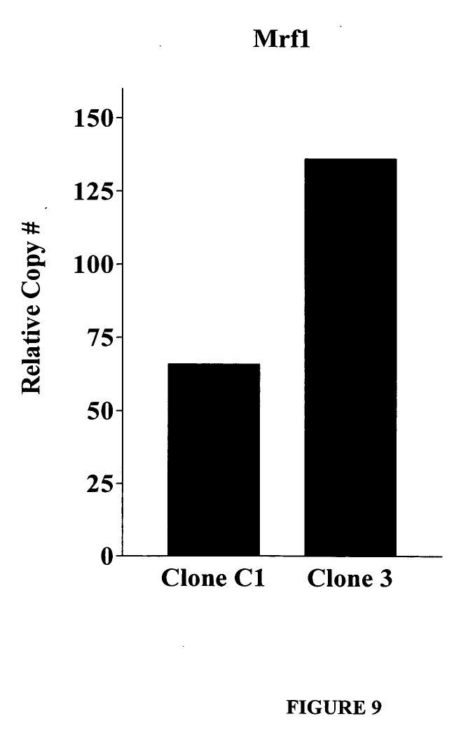

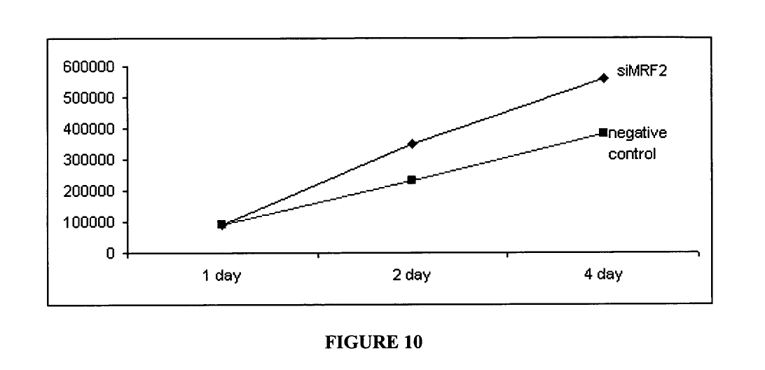

Finally, another embodiment uses short interfering RNA to Mrf-2 to control the expression of Mrf-2 in vivo in a cell. The oligonucleotides sequences specific for Mrf-2, 5′GATCCCGCCTTCTTGGTGGCACTTTTCAAGAGAAAAGTGCCACCAAGA AGGCTTTTTTGGAAA-3′ (SEQ ID NO: 5) and 5′-AGCTTTTCCAAAAAAGCCTTCTTGGTGGCACTTTTCTCTTGAAAAAGTGCC ACCAAGAAGGCGG-3′ (SEQ ID NO: 6), were synthesized and annealed. The siRNA expression vector pSilencer-U6Hygromycin for Mrf-2 was constructed by inserting the annealed DNA at a BamH1/HindIII site. A pSilencer vector (Ambion, Austin, Tex.) that expresses a hairpin siRNA with limited homology to any known sequences in the human, mouse, and rat genomes was used as a negative control. Mrf-2 siRNA expression vector and negative control were stably transfected into prostate cell line DU145 by reagent Siport-xp1 (Ambion). The RNA was isolated from the stable cloned cell lines for quantitative expression of Mrf-1 and Mrf-2 by real-time PCR. FIG. 8 shows that the Mrf-2 expression in cells with siRNA targeted to Mrf-2 was about five times less than control cells. When the experiment using siRNA targeting Mrf-2 was used on Mrf-1 cells, surprisingly, the expression of Mrf-1 was about twice in the targeted cells as in the control cells. Thus, downregulating the expression of Mrf-2 upregulates the expression of Mrf-1. This finding may be used to control Mrf-1 in cells. The cell proliferation assay was performed by growing the cells without serum and hygromycin selection with the specified time. Cells were counted with a Coulter counter. FIG. 10 shows the results of the cell proliferation assay.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

### CITED REFERENCES

[1] S. Gregory, R. D. Kortschak, B. Kalionis, R. Saint, “Characterization of the dead ringer gene identifies a novel, highly conserved family of sequence-specific DNA binding proteins,” Mol. Cell. Biol. 16 (1996) 792-799.

[2] R. F. Herrscher, M. H. Kaplan, D. L. Lelsz, C. Das, R, Scheuermann, P. W. Tucker, “The immunoglobulin heavy-chain matrix-associating regions are bound by Bright: a B cell-specific trans-activator that describes a new DNA-binding protein family,” Genes Dev. 9 (1995) 3067-3082.

[3] R. D. Kortschak, P. W. Tucker, R. Saint, “ARID proteins come in from the desert,” Trends Biochem. Sci. 25 (2000) 294-299.

[4] Y.-C Yuan, R. H. Whitson, Q. Liu, K. Itakura, Y. Chen, “A novel DNA-binding motif shares structural homology to DNA replication and repair nucleases and polymerases,” Nature Struct. Biol. 5 (1998) 959-964.

[5] J. Iwahara, R. T. Clubb, “Solution structure of the DNA binding domain from Dead ringer, a sequence-specific AT-rich interaction domain (ARID),” EMBO J. 18 (1999) 6084-6094.

[6] T. Shandala, R. D. Kortschak, S. Gregory, R. Saint, “The Drosophila dead ringer gene is required for early embryonic patterning through regulation of argos and buttonhead expression,” Development 126 (1999) 4341-4349.

[7] R. H. Whitson, T. Huang, K. Itakura, “The novel Mrf-2 DNA-binding domain recognizes a five-base core sequence through major and minor-groove contacts.” Biochem. Biophys. Res. Commun. 258 (1999) 326-331.

[8] P. B. Dallas, S. Pacchione, D. Wilsker, V. Bowrin, R. Kobayashi, E. Moran, “The human SWI/SNF complex protein p270 is an ARID family member with nonsequence-specific DNA binding activity,” Mol. Cell. Biol. 20 (2000) 3137-3146.

[9] M. Vazquez, L. Moore, J. A. Kennison, “The trithorax group gene osa encodes an ARID domain protein that genetically interacts with the Brahma chromatin remodeling factor to regulate transcription,” Development 126 (1999) 733-742.

[10] T. H. Huang, T. Oka, T. Asai, T. Okada, B. W. Merrills, P. N. Gertson, R. H. Whitson, K. Itakura, “Repression by a differentiation-specific factor of the human cytomegalovirus enhancer,” Nucleic Acids Res. 24 (1996)1695-1701.

[11] M. Rossel, M. Capecchi, “Mice mutant for both Hoxa1 and Hoxb1 show extensive remodeling of the hindbrain and defects in craniofacial development,” Development 126 (1999) 5027-5040.

[12] B. Hogan, R. Beddington, F. Constantine, E. Lacy, “Manipulating the mouse embryo,” New York, Cold Spring Harbor Press (1994).

[13] R. R. Wolfe, “Tracers in metabolic research; Radioisotope and stable isotope/mass spectrometry methods,” New York, Alan R. Liss, Inc. (1984).

[14] J. M. Culebras, F. D. Moore, “Total body water and the exchangeable hydrogen I. Theoretical calculation of nonaqueous exchangeable hydrogen in man,” Am. J. Physiol., Reg. Integrative Comp. Physiol. 232 (1977) R54-R59.

[15] S. Cinti, M. C. Zingaretti, R. Cancello, E. Ceresi, P. Farrara, “Morphologic techniques for the study of brown adipose tissue and white adipose tissue,” Meth. Mol. Biol. 155 (2001) 21-52.

[16] K. M. Downs, T. Davies, “Staging of gastrulating mouse embryos by morphological landmarks in the dissecting microscope,” Development 118 (1993) 1255-1266.

[17] T. Tanaka, N. Yoshida, T. Kishimoto, S. Akira, “Defective adipocyte differentiation in mice lacking C/EBPβ and/or C/EBPδ, “EMBO J. 16 (1997) 7432-7443.

[18] N.-D. Wang, M. J. Finegold, A. Bradley, C. N. Ou, S. A. Abdelsayed, M. D. Wilde, L. R. Tayler, D. R. Wilson, G. J. Darlington, “Impaired energy homeostasis in C/EBPa knockout mice,” Science 269 (1995)1108-1112.

[19] W.-C. Yeh, Z. Cao, M. Classon, S. L. McKnight, “Cascade regulation of terminal adipocyte differentiation by three members of the C/EBP family of leucine zipper proteins,” Genes Dev. 9 (1995)168-169.

[20] T. Yamauchi, Y. Oieke, J. Kamon, H. Waki, K. Komeda, A. Tsuchida, Y. Date, M.-X. Li, H. Miki, Y. Akanuma, R. Nagai, S. Kimura, T. Saheki, M. Nakazato, T. Naitoh, K. Yamamura, T. Kadowaki, “Increased insulin sensitivity despite lipodystrophy in Crebbp heterozygous mice,” Nature Genetics 30 (2002) 221-226.

[21] Whitson R. H., Huang T., Itakura K., Tsark W., “Neonatal mortality and leanness in mice lacking the ARID transcription factor Mrf-2,” Biochem. Biophys. Res. Commun. 312:997-1004 (2003).

[22] M. Watanabe, M. D. Layne, C.-M. Hsieh, K. Maemura, S. Gray, M. E. Lee, M. K. Jain, “Regulation of smooth muscle cell differentiation by AT-Rich interaction domain transcription factors Mrf-2α and Mrf-2β,” Circulation Res. 9 (2002) 382-389.

[23] Kadenbach B. Intrinsic and extrinsic uncoupling of oxidative phosphorylation. Biochim Biophys Acta. 1604:77-94 (2003).

[24] Tiraby C, Tavenier G, Lefort C, Larrouy D, Ricquier D, Langin D. Acquirement of brown fat cell features by human white adipocytes. J. Biol. Chem. 278:33370-33376 (2003).

[25] Gregoire F M. Adipocyte differentiation: from fibroblast to endocrine cell. Exp. Biol. Med. 226:9971002 (2001).

[26] Umek R M, Friedman A D, McKnight S L. CCAAT/enhancer binding protein: a component of a differentiation switch. Science 251:288-292 (1991).

[27] Guerra C, Koza R A, Yamashita H, Walsh K and Kozak L P. Emergence of brown adipocytes in white fat in mice is under genetic control. Effects on body weight and adiposity. J Clin Invest 102:412-420 (1998).

[28] Ntambi J M, Miyazaki M, Stoer J P, Lan H, Kendziorski C M, Yandell, B S, Song Y, Cohen P, Friedman J M, Attie, A D. Loss of stearoyl-CoA desaturase-1 function protects mice against adiposity. Proc Natl Acad Sci U S A. 99:11482-11486 (2002).

[29] De Silva B., What syndrome is this? Rubenstein-Taybi syndrome. Pediatric Dermatol. 19:177-179 (2002).

[30] Yamauchi T, Oike Y, Kamon J, Waki H, Komeda K, Tsuchida A, Date Y, Li M X, Miki H, Akanuma Y, Nagai R, Kimura S, Saheki T, Nakazato M, Naitoh T, Yamamura K, Kadowaki T, Increased insulin sensitivity despite lipodystrophy in Crebbp heterozygous mice. Nat Genet. 30:221-226 (2002).

[31] Abdel-Malek Z A, Scott M C, Furumura M, Lamoreux M L, Ollmann M, Barsh G S, Hearing V J. The melanocortin 1 receptor is the principal mediator of the effects of agouti signaling protein on mammalian melanocytes. J Cell Sci. 114:1019-1024 (2001).

[32] Bergen H T, Mizuno T M, Taylor J, Mobbs C V. Hyperphagia and weight gain after gold-thioglucose: relation to hypothalamic neuropeptide Y and proopiomelanocortin. Endocrinology. 139:4483-8 (1998).

[33] Morris M J, Tortelli C F, Filippis A, Proietto J Reduced BAT function as a mechanism for obesity in the hypophagic, neuropeptide Y deficient monosodium glutamate-treated rat. Regul. Pept. 75-76:441-447 (1998).

[34] Pelleymounter M A, Cullen M J, Baker M B, Hecht R, Winters D, Boone T, Collins F. Effects of the obese gene product on body weight regulation in ob/ob mice. Science 269:540-543 (1995).

[35] Halaas J L, Gajiwala K S, Maffei M, Cohen S L, Chait B T, Rabinowitz D, Lallone R L, Burley S K, Friedman J M. Weight-reducing effects of the plasma protein encoded by the obese gene. Science 269:543-456 (1995).

[36] Lai T., Kim E., Nguyen M., Ortiz A., Tachibana M., Whitson R. H., Lyons C., Law C., “Craniofacial Analysis of Mice Deficient in Mrf-2” presented as a poster at the International Association of Dental Research Conference, March (2004).

[37] Tartaglia L A, Dembski M, Weng X, Deng N, Culpepper J, Devos R, Richards G J, Campfield L A, Clark F T, Deeds J. Identification and expression cloning of a leptin receptor. Cell 83:1263-1271 (1996).

[38] Chen H, Charlat O, Tartaglia L A, Woolf E A, Weng X, Ellis S J, Lakey N D, Culpepper J, Moore K J, Breitbart R E, Duyk G M, Tepper R I, Morgenstern J P. Evidence that the diabetes gene encodes the leptin receptor: identification of a mutation in the leptin receptor gene in db/db mice. Cell 84:491-495 (1996).

[39] Breslow M J, Min-Lee K, Brown D R, Chacko V P, Palmer D, Berkowitz D E Effect of leptin deficiency on metabolic rate in ob/ob mice. Am J Physiol. 276:E443-9 (1999).

[40] Hahm S, Fekete C, Mizuno T M, Windsor J, Yan H, Boozer C N, Lee C, Elmquist J K, Lechan R M, Mobbs C V and Salton S R J. VGF is required for obesity induced by diet, gold thioglucose treatment, and agouti and is differentially regulated in pro-opiomelanocortin- and neuropeptide Y-containing arcuate neurons in response to fasting. J Neurosci 22:6929-6938 (2002).

[41] Bluher, M., Michael, M. D., Peroni, O. D., Ueki, K, Carter, N., Kahn, B. B. and Kahn, C. R. (2002) Adipose Tissue Selective Insulin Receptor Knockout protects against obesity and obesity-related glucose intolerance. Developmental Cell 3:25-38.

### OTHER REFERENCES

Campfield L A, Smith F J, Guisez Y, Devos R, Burn P. Recombinant mouse OB protein: Evidence for a peripheral signal linking adiposity and central neural networks. Science 269:546-549 (1995).

Kortschak R D, Tucker P W and Saint R. ARID proteins come in from the desert. Trends Biochem. Science 25: 294-299, (2000).

Lahoud M H, Ristevski S, Venter D J, Jermiin L S, Bertoncello I, Zavarsek S, Hasthorpe S, Drago J, de Kretser D, Hertzog P J, Kola I, “Gene targeting of Desrt, a novel ARID class DNA-binding protein, causes growth retardation and abnormal development of reproductive organs,” Genome Res. 11 1327-1334 (2001).

1. A method of determining whether a subject is predisposed to leanness, craniofacial defects, inborn errors in metabolism, and/or is resistant to diabetes, comprising screening the subject for Mrf-2, wherein
(a) the presence of a nonfunctional Mrf-2 gene or absence of Mrf-2 protein indicates that the subject is predisposed to leanness, craniofacial defects, inborn errors in metabolism, and is resistant to diabetes; and
(b) the presence of a functional Mrf-2 gene or presence of Mrf-2 protein indicates that the subject is not is predisposed to leanness, craniofacial defects, inborn errors in metabolism, and is not resistant to diabetes.

2. The method of claim 1, wherein, if the screening is conducted for the Mrf-2 gene, the screening is accomplished by extracting nucleic acid from the subject, amplifying the portion of the nucleic acid encoding the Mrf-2 gene, and determining whether the Mrf-2 gene is functional or nonfunctional.

3. The method of claim 2, wherein a functional Mrf-2 gene has the form of Mrf-2^{+/+} or Mrf-2^{±} and a nonfunctional Mrf-2 gene has the form of Mrf-2^{-/-} or is a deletion, truncation, or mutation of Mrf-2^{+/+} or Mrf-2^{±}.

4. The method of claim 2, wherein amplifying the nucleic acid comprises using polymerase chain reaction and wherein the nucleic acid is DNA.

5. The method of claim 4, wherein a reaction mixture used for the polymerase chain reaction comprises DNA polymerase and primers complementary to Mrf-2 DNA.

6. The method of claim 2, wherein the subject is a mammal.

7. The method of claim 6, wherein the mammal is a human.

8. The method of claim 1, wherein the screening is conducted prenatally.

9. The method of claim 1, wherein the screening is conducted postnatally.

10. The method of claim 1, wherein, if the screening is conducted for the Mrf-2 protein, the screening is accomplished by extracting a sample from a subject that would contain the protein if the protein were present and testing for the presence of Mrf-2.

11. The method of claim 10, wherein Western blotting is used to detect whether Mrf-2 is present.

12. A method of screening for a modulator of adipocyte maturation or adipocyte function using Mrf-2 protein or nucleic acid, comprising:
(a) exposing Mrf-2 protein or nucleic acid to a test compound;
(b) determining whether the test compound binds to Mrf-2 protein or nucleic acid; and
(c) if the test compound binds, selecting the test compound as a possible modulator of Mrf-2.

13. The method of claim 12, wherein the nucleic acid is DNA.

14. The method of claim 12, wherein the modulator activates or enhances Mrf-2 activity.

15. The method of claim 12, wherein the modulator inhibits Mrf-2 activity.

16. The method of claim 12, wherein the binding is detected by high-throughput screening.

17. The method of claim 12, wherein the Mrf-2 protein used in the screening has its C-terminal portion is truncated to within 100 amino acids of its DNA-binding domain.

18. The method of claim 17, wherein the Mrf-2 protein used in the screening consists of its DNA-binding domain.

19. The method of claim 12, wherein the modulator is a Mrf-2-specific antibody, a Mrf-2-specific small molecule, or Mrf-2-specific siRNA.

20. The method of claim 12, wherein the modulator binds directly to Mrf-2.

21. The method of claim 12, wherein the modulator binds to a Mrf-2 substrate, binding partner, or cofactor.

22. A method of screening for a modulator of adipocyte maturation or adipocyte function using Mrf-2 protein or nucleic acid, comprising:
(a) exposing Mrf-2 protein or nucleic acid to a test compound;
(b) determining whether the test compound binds to Mrf-2 protein or nucleic acid;
(c) if the test compound binds, selecting the test compound as a possible modulator of Mrf-2 activity;
(d) determining the effect of the possible modulator on adipocyte maturation in mouse embryo fibroblasts derived from Mrf-2^{-/-}, Mrf-2⁺⁻ and Mrf-2^{+/+} embryos; and
(e) if the possible modulator affects adipocyte maturation, determining the effect of the possible modulator on fat accumulation in adult Mrf-2^{-/-}, Mrf-2⁺⁻ and Mrf-2^{+/+} mice.


SEQUENCE LISTING (6 sequences)

SEQ ID NO: 1 - DNA, 23 bp, Artificial
  PCR Primer
cgcttgggtggagaggctattcg

SEQ ID NO: 2 - DNA, 23 bp, Artificial
  PCR Primer
cggcaggagcaaggtgagatgac

SEQ ID NO: 3 - DNA, 24 bp, Artificial
  PCR Primer
tgcatagaatgaatgaccctggtc

SEQ ID NO: 4 - DNA, 22 bp, Artificial
  PCR Primer
cggaagtggacagatggaatgg

SEQ ID NO: 5 - DNA, 64 bp, Artificial
  Synthesized oligonucleotide
gatcccgccttcttggtggcactttttcaagagaaaagtgccaccaagaaggcttttttggaaa

SEQ ID NO: 6 - DNA, 64 bp, Artificial
  Synthesized oligonucleotide
agcttttccaaaaaagccttcttggtggcacttttctcttgaaaaagtgccaccaagaaggcgg